SEQUENCE LISTING (12 sequences)

SEQ ID NO: 1 - DNA, 39 bp, Artificial Sequence
  synthetic primer
ggtgggggaggtgaattcatggtgagcaagggcgaggag

SEQ ID NO: 2 - DNA, 39 bp, Artificial Sequence
  synthetic primer
gtggtcagatcctcgagcttgtacagctcgtccatgccg

SEQ ID NO: 3 - DNA, 30 bp, Artificial Sequence
  synthetic primer
aattggttcctggtcgaccgatgatccgcg

SEQ ID NO: 4 - DNA, 30 bp, Artificial Sequence
  synthetic primer
tcgacgcggatcatcggtcgaccaggaacc

SEQ ID NO: 5 - DNA, 18 bp, Artificial Sequence
  synthetic primer
acatattcgagatttgac

SEQ ID NO: 6 - DNA, 69 bp, Artificial Sequence
  synthetic primer
tgcggccgctgccctaggccctccgccacctccatgaccgaaaccgtatgtggtcagacccttttctgg

SEQ ID NO: 7 - DNA, 63 bp, Artificial Sequence
  synthetic primer
ggtttcggtcatggaggtggcggagggcctagggcagcggccgcaggattaacgacttatgga

SEQ ID NO: 8 - DNA, 50 bp, Artificial Sequence
  synthetic primer
aaaaggtcgactaatacgactcactataggttaaaacagctctggggttg

SEQ ID NO: 9 - DNA, 56 bp, Artificial Sequence
  synthetic primer
gggggaagcttaggccttttttttttttttttttttcctccgaattaaagaaaaat

SEQ ID NO: 10 - DNA, 21 bp, Artificial Sequence
  synthetic primer
gccctgggctcttgattctgt

SEQ ID NO: 11 - DNA, 18 bp, Artificial Sequence
  synthetic primer
gaaggcgattcgttgtac

SEQ ID NO: 12 - DNA, 18 bp, Artificial Sequence
  synthetic primer
cttgattcagccactaag

(19) United States
(12) Patent Application Publication
(10) Pub. No.: US 2003/0068332 A1
(43) Pub. Date: Apr. 10, 2003

(54) Replication-competent recombinant virus and methods of use thereof

(76) Inventors: Raul Andino-Pavlovsky, San Francisco, CA (US); Shane Crotty, Atlanta, GA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/104,192

(22) Filed: Mar. 22, 2002

Related U.S. Application Data
(60) Provisional application No. 60/279,553, filed on Mar. 28, 2001.

(51) Int. Cl. C12P 21/06; A61K 39/12; A61K 39/21; A61K 39/13

(52) U.S. Cl. 424/199.1; 424/204.1; 424/217.1; 424/208.1; 424/207.1; 435/69.1

(57) ABSTRACT

The present invention provides a population of live attenuated recombinant replication-competent viruses, which population comprises at least two member viruses, each of the member viruses comprising a nucleotide sequence encoding a different antigenic polypeptide from a source other than a parent virus from which the recombinant virus was derived. When a eukaryotic cell of a mammalian host is infected with a member of the population, the nucleotide sequence is expressed, the antigenic polypeptide is produced, and elicits an immune response. The invention further provides compositions, including immunogenic compositions, comprising a subject virus population. The invention further provides methods of eliciting an immune response to a polypeptide in an individual, involving administering a subject virus population. The invention further provides devices for use in eliciting an immune response.

### CROSS-REFERENCE

This application claims the benefit of U.S. Provisional Patent Application No. 60/279,553, filed Mar. 28, 2001, the contents of which are incorporated herein by reference in their entirety.

### STATEMENT REGARDING FEDERALLY SPONSORED RESEARCH

The U.S. government may have certain rights in this invention pursuant to grant no. AI36178 awarded by the Public Health Service.

### FIELD OF THE INVENTION

This invention is in the field of recombinant viral vectors and vaccines.

### BACKGROUND OF THE INVENTION

Live viral vectors are attractive candidates for use in compositions for inducing an immune response in a subject for several reasons. For example, the Sabin live poliovirus vaccine is one of the best human vaccines in the world. It produces long lasting immunity and herd immunity; it is very safe and easy to manipulate experimentally; it has a proven safety and efficacy record in over 1 billion vaccines; it is inexpensive to produce and distribute in developing countries; and perhaps most importantly, it produces a potent mucosal immune response.

A variety of picomaviral vectors have been explored as potential vaccine vectors. Indeed, live viral vectors are leading candidates in the hunt for a potential AIDS vaccine. Several viral vectors have shown promise in that they protect monkeys against highly virulent strains of SIV (a primate AIDS vaccine model system). A number of other live viral vector systems are in earlier testing phases of vaccine development. Viral vectors are also being explored as potential tumor vaccines, and poliovirus vectors, picomavirus vectors, and vaccinia vectors have proven efficacious in mouse tumor model systems. A picomaviral vaccine vector is described in U.S. Pat. No. 5,965,124.

Two of the more significant restrictions of the live picomavirus vectors are the insert size limit (which is approximately 300-400 amino acids), and the tendency of recombinant polioviruses to generate deletions in the insert after several rounds of replication, such that viruses having deletions in the insert become prevalent in the viral population after multiple passages.

Libraries of nucleic acid molecules isolated from pathogens and inserted into plasmid expression vectors have been described. See, e.g., U.S. Pat. No. 5,703,057. However, the plasmids, which replicate little, if at all, in mammalian cells, provide for a limited amount of protein production and only for a limited amount of time.

Thus, there is a need in the art for improved compositions for generating or enhancing an immune response to pathogens such as human immunodeficiency virus or other microbial pathogens. The present invention addresses this need.

### Literature

U.S. Pat. No. 5,703,057; U.S. Pat. No. 5,965,124; U.S. Pat. No. 6,245,532; U.S. Pat. No. 6,328,975; U.S. Pat. No. 6,294,176; Crotty et al. (2001) J. Virol. 75:7435-7452; Altmeyer et al. (1995) J. Virol. 69:4193-3196; Andino et al. (1994) Science 265:3193-3196; Crotty et al. (1999) J. Virol. 73:9485-9495; Hanke et al. (1999) Vaccine 17:589-596; Hirsch et al. (1996) J. Virol. 70:3741-3752; Morrow et al. (1999) Curr. Topics Microbiol. Immunol. 236:255-273; Murphy et al. (2000) J. Virol. 74:7745-7754; Robinson et al. (1999) Nat. Med. 5:526-534; Ruprecht (1999) Immunol. Rev. 170:135-149; Tang et al. (1997) J. Virol. 71:7841-7850; Yasutomi et al. (1995) J. Virol. 69:2279-2284; Yim et al. (1996) Virol. 218:61-70.

### SUMMARY OF THE INVENTION

The present invention provides a population of live attenuated recombinant replication-competent viruses, which population comprises at least two member viruses, each of the member viruses comprising a nucleotide sequence encoding a different antigenic polypeptide from a source other than a parent virus from which the recombinant virus was derived. When a eukaryotic cell of a mammalian host is infected with a member of the population, the nucleotide sequence is expressed, the antigenic polypeptide is produced, and elicits an immune response. The invention further provides compositions, including immunogenic compositions, comprising a subject virus population. The compositions are useful to elicit an immune response in an individual to antigenic polypeptides. The invention further provides methods of eliciting an immune response to a polypeptide in an individual, involving administering a subject virus population. The invention further provides devices for use in eliciting an immune response.

### FEATURES OF THE INVENTION

In one aspect, the invention provides a population of live attenuated recombinant replication-competent viruses, which population comprises at least two member viruses. Each of the member viruses comprises a nucleotide sequence encoding a different exogenous antigenic polypeptide from a source, such as a pathogenic organism or a tumor, other than a parent virus from which the recombinant virus was derived. The nucleotide sequences encoding the exogenous polypeptides are capable of being expressed in a eukaryotic cell of a mammalian host organism.

In another aspect, the invention provides a population of live attenuated recombinant replication-competent polioviruses, which population comprises at least two member viruses. Each of the member viruses comprises a nucleotide sequence encoding a different exogenous antigenic polypeptide from a source, such as a pathogenic organism or a tumor, other than poliovirus. The nucleotide sequences encoding the exogenous polypeptides are capable of being expressed in a eukaryotic cell of a mammalian host organism.

In some embodiments, the exogenous antigenic polypeptides are included within polyprotein precursors which are proteolytically processed to release the antigenic polypeptides.

In some embodiments, the population expresses at least two different antigenic polypeptides, or fragments thereof, from a pathogenic organism or a tumor. In other embodiments, the population expresses at least three different antigenic polypeptides, or fragments thereof, from a pathogenic organism or a tumor. In other embodiments, the population expresses at least four different antigenic polypeptides, or fragments thereof, from a pathogenic organism or TAA. In still other embodiments, the population expresses more than four different antigenic polypeptides, or fragments thereof, from a pathogenic organism or a tumor. The encoded polypeptides are from about 4 amino acids to about 400 amino acids in length.

In some embodiments, the antigenic polypeptides are human immunodeficiency virus (HIV) polypeptides or fragments thereof. In some of these embodiments, the HIV is HIV type-1. In some embodiments, the at least two different antigenic polypeptides, or fragments thereof, are selected from the group consisting of gag, env, pol and nef polypeptides from HIV. In some of these embodiments, the population comprises nucleotide sequences encoding overlapping fragments of the gag, env, pol and nef polypeptides and each fragment has a length from about four amino acids to about 400 amino acids, from about four amino acids to about 100 amino acids, or from about 100 amino acids to about 250 amino acids.

In some embodiments, the population expresses from about 10% to about 25% of the antigenic polypeptides from the pathogenic organism or tumor. In other embodiments, the population expresses from about 25% to about 50% of the antigenic polypeptides from the pathogenic organism or tumor. In still other embodiments, the population expresses from about 50% to about 90% of the antigenic polypeptides from the pathogenic organism or tumor.

In some embodiments, the recombinant virus is a DNA virus. In other embodiments, the recombinant virus is an RNA virus. In some embodiments, the recombinant RNA virus a picomavirus, e.g., a poliovirus.

In some embodiments, the encoded antigenic polypeptides those of a pathogenic virus, e.g., a human immunodeficiency virus, or an influenza virus.

In another aspect, the invention provides a composition comprising a subject population and a pharmaceutically acceptable carrier. In many embodiments, the composition is an immunogenic composition.

In another aspect, the invention provides a method of eliciting an immune response in a mammalian host to an antigenic polypeptide. The method generally involves administering a first population of live attenuated recombinant poliovirus to a mammalian host, where the first population is in a first strain of poliovirus, wherein the first population comprises at least two member viruses, wherein each of said member virus comprises a nucleotide sequence encoding a different exogenous antigenic polypeptide, wherein said administering provides for infection of a mammalian host cell and expression of the antigenic polypeptides, and wherein expression of the antigenic polypeptides results in induction of an immune response in the host to the antigenic polypeptides. In some embodiments, the immune response is a mucosal immune response. In some embodiments, the methods involve administering a composition of the invention to the host.

In some embodiments, the methods further involve administering to a host a second population of live attenuated recombinant poliovirus, wherein the second population of recombinant poliovirus is in a second strain of poliovirus, wherein the second population comprises at least two member viruses, wherein each of said member virus comprises a nucleotide sequence encoding a different antigenic polypeptide, the second population being administered after administration of the first population, wherein an immune response to the antigenic polypeptides is elicited in the host.

In some embodiments, the second population is administered at a time period of from about 1 day to about 1 week after administration of the first population. In other embodiments, the second population is administered at a time period of from about 1 week to about 4 weeks after administration of the first population. In other embodiments, the second population is administered at a time period of from about 1 month to about 6 months, or longer, after administration of the first population.

In a specific embodiment, the first population is in the Sabin-1 strain of poliovirus and the second population is in the Sabin-2 strain of poliovirus.

In another aspect, the invention provides a device for administering an immunogenic composition comprising a subject replication-competent recombinant virus population. In some embodiments, the device comprises a container that contains therein a subject immunogenic composition. In some embodiments, the device comprises a syringe and a needle.

In another aspect, the invention provides a method of making a population of live, attenuated, recombinant replication-competent viruses. The method generally involves inserting a plurality of nucleic acids into a genome of a virus or a portion thereof which is capable of replicating in a desired host organism, said plurality of nucleic acids encoding a plurality of antigenic polypeptides from a pathogenic organism, forming a population of recombinant virus constructs; and obtaining a population of live attenuated recombinant replication-competent viruses from said population of recombinant virus constructs.

### BRIEF DESCRIPTIONS OF THE DRAWINGS

FIGS. 1A depicts recombinant Sabin poliovirus vector; and FIG. 1B depicts a schematic representation of antigens encoded by members of a recombinant poliovirus library.

FIG. 2 presents a timeline of vaccination and challenge.

FIGS. 3A and 3B are graphs depicting serum anti-SIV antibodies generated in response to administration of a recombinant poliovirus population. Seven cynomolgus macaques were inoculated intranasally with SabRV1-SIV at week 0 and 2 (indicated by open triangle below x axis) and boosted intranasally at week 19 and 21 with SabRV2-SIV (indicated by solid triangle below x axis).

FIGS. 4A and 4B are graphs depicting rectal anti-SIV antibodies generated in response to administration of a recombinant poliovirus population. Monkeys are labeled as in FIGS. 3A and 3B.

FIGS. 5A and 5B are graphs depicting vaginal anti-SIV antibodies generated in response to administration of a recombinant poliovirus population. Monkeys are labeled as in FIGS. 3A and 3B.

FIGS. 6A and 6B are graphs depicting SIV-specific cytotoxic T lymphocytes generated after immunization with SabRV1-SIV (FIG. 8A) and SabRV2-SIV (FIG. 8B).

FIG. 7 presents graphs depicting serum anti-SIV IgG antibody responses post challenge. Vaccinated monkeys (right panel) are indicated by symbols used in previous figures. Results with control monkeys are shown in the left panel.

FIG. 8 presents graphs depicting SIV RNA loads in plasma post-challenge. Symbols for vaccinated monkeys are as in previous figures. Symbols for control monkeys are as in FIG. 7. Vaccinated monkeys 27244 and 27270 were never positive of SIV RNA and appear completely protected.

FIGS. 9A-C are graphs depicting clinical outcomes of vaginal challenge with SlV_{mac}251. Symbols for vaccinated and control monkeys are as in FIG. 7. FIG. 9A: Post-challenge CD4⁺T lymphocyte counts; FIG. 9B: Body weight; and FIG. 9C: Mortality curve.

### DEFINITIONS

The terms “polynucleotide” and “nucleic acid”, used interchangeably herein, refer to a polymeric forms of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “oligopeptide,” “polypeptide,” “polyprotein,” and “protein” are used interchangeably herein, and refer to a polymeric form of amino acids of any length, which can include coded and non-coded amino acids, chemically or biochemically modified or derivatized amino acids, and polypeptides having modified peptide backbones.

The term “recombinant viral polyprotein precursor,” as used herein, refers to a viral polyprotein precursor that contains inserted therein an exogenous protein, e.g., a protein that is not normally part of the wild-type viral polyprotein precursor found in nature.

“Recombinant,” as used herein, means that a particular DNA sequence is the product of various combinations of cloning, restriction, and/or ligation steps resulting in a construct having a structural coding sequence distinguishable from homologous sequences found in natural systems. Generally, DNA sequences encoding the structural coding sequence can be assembled from cDNA fragments and short oligonucleotide linkers, or from a series of oligonucleotides, to provide a synthetic gene which is capable of being expressed in a recombinant transcriptional unit. Such sequences can be provided in the form of an open reading frame uninterrupted by internal nontranslated sequences, or introns, which are typically present in eukaryotic genes. Genomic DNA comprising the relevant sequences could also be used. Sequences of non-translated DNA may be present 5′ or 3′ from the open reading frame, where such sequences do not interfere with manipulation or expression of the coding regions. Thus, e.g., the term “recombinant” polynucleotide or nucleic acid refers to one which is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

By “construct” is meant a recombinant nucleic acid, generally recombinant DNA, that has been generated for the purpose of the expression of a specific nucleotide sequence(s), or is to be used in the construction of other recombinant nucleotide sequences.

Similarly, a “recombinant polypeptide” or “recombinant polyprotein” refers to a polypeptide or polyprotein which is not naturally occurring, or is made by the artificial combination of two otherwise separated segments of amino acid sequences. This artificial combination may be accomplished by standard techniques of recombinant DNA technology, such as described above, i.e., a recombinant polypeptide or recombinant polyprotein may be encoded by a recombinant polynucleotide. Thus, a recombinant polypeptide or recombinant polyprotein is an amino acid sequence encoded by all or a portion of a recombinant polynucleotide.

As used herein, the term “exogenous polypeptide” refers to a polypeptide that is not normally associated with a parent virus in nature. The term “exogenous polypeptide” encompasses fragments of a polypeptide, including antigenic fragments, e.g., fragments containing one or more antigenic determinants.

The term “immunologically active” or “immunogenic” refers to the capability of the natural, recombinant, or synthetic peptide to induce a specific humoral and/or cellular immune response in a mammal. As used herein, “antigenic amino acid sequence,” “antigenic polypeptide,” or “antigenic peptide” means an amino acid sequence that, either alone or in association with an accessory molecule (e.g. a class I or class II major histocompatibility antigen molecule), can elicit an immune response in a mammal. Thus, the term “antigenic polypeptide” encompasses full-length polypeptides, and antigenic fragments.

As used herein, “an immune response” is meant to encompass cellular and/or humoral immune responses that are sufficient to inhibit or prevent infection, or prevent or inhibit onset of disease symptoms caused by a microbial organism, particularly a pathogenic microbial organism, and/or to inhibit, reduce, or prevent proliferation of a tumor cell, and/or to reduce tumor cell numbers or tumor mass, and/or to reduce the likelihood that a tumor will form. The terms “mucosal immune response” and “mucosal immunity” is a term well understood in the art, and refers to an immune response characterized by production of secretory IgA in mucosal tissues such as gastrointestinal tract tissues, including rectal tissues; vaginal tissues; and tissues of the respiratory tract.

The term “tumor-associated antigen” is a term well understood in the art, and refers to surface molecules that are differentially expressed in tumor cells relative to non-cancerous cells of the same cell type. As used herein, “tumor-associated antigen” includes not only complete tumor-associated antigens, but also epitope-comprising portions (fragments) thereof. A tumor-associated antigen (TAA) may be one found in nature, or may be a synthetic version of a TAA found in nature, or may be a variant of a naturally-occurring TAA, e.g., a variant has enhanced immunogenic properties.

The terms “antigen” and “epitope” are well understood in the art and refer to the portion of a macromolecule which is specifically recognized by a component of the immune system, e.g., an antibody or a T-cell antigen receptor. Epitopes are recognized by antibodies in solution, e.g., free from other molecules. Epitopes are recognized by T-cell antigen receptor when the epitope is associated with a class I or class II major histocompatibility complex molecule. A “CTL epitope” is an epitope recognized by a cytotoxic T lymphocyte (usually a CD8⁺ cell) when the epitope is presented on a cell surface in association with an MHC Class I molecule.

As used herein the term “isolated” is meant to describe a compound of interest (e.g., a recombinant virus, a peptide, etc.) that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, preferably 75% free, and most preferably 90% free from other components with which it is naturally associated.

By “subject” or “individual” or “host” or “patient,” which terms are used interchangeably herein, is meant any subject, particularly a mammalian subject, for whom diagnosis or therapy is desired, particularly humans. Other subjects may include cattle, dogs, cats, guinea pigs, rabbits, rats, mice, non-human primates, horses, and so on. Of particular interest are those subjects susceptible to infection by a virus, e.g., a picomavirus (e.g., a poliovirus), e.g., subjects who can support poliovirus replication.

A “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids and tissue samples.

The terms “treatment,” “treating,” and the like are used herein to generally refer to obtaining a desired pharmacologic or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, e.g., a human, and includes: (a) preventing the disease from occurring in a subject which may be predisposed to the disease but has not yet been diagnosed as having it, e.g., reducing the risk that an individual will develop the disease, reducing the severity of a disease symptom; (b) inhibiting the disease, i.e., arresting its development; and (c) relieving the disease, i.e., causing regression of the disease. In some embodiments, the invention is directed toward treating patients with cancer. In these embodiments, “treatment” can include reducing or inhibiting tumor cell growth, eliminating a tumor, reducing metastasis, reducing or inhibiting tumor cell proliferation, reducing tumor cell mass, reducing tumor cell number, and reducing the probability that a tumor will form. In other embodiments, the invention is directed toward reducing or preventing a disease caused by a pathogenic organism. In these embodiments, “treatment” can include reducing the severity of a disease after infection with a pathogenic organism.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. Cancerous cells can be benign or malignant.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a recombinant virus” includes a plurality of such viruses and reference to “the polypeptide” includes reference to one or more polypeptides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

The present invention provides a population of replication competent recombinant viruses comprising a plurality of members. The replication-competent virus can be any virus capable of replicating in a subject. Of particular interest are replication-competent recombinant viral populations that exhibit attenuated pathogenicity. The population of replication-competent recombinant viruses comprises at least two members, each of which comprises a nucleotide sequence encoding a different antigenic polypeptide not encoded by the parent virus from which the recombinant virus is derived. The nucleotide sequences encoding the different antigenic polypeptides are expressed in a host cell infected by the recombinant virus, and the antigenic polypeptides elicit a host immune response.

In some embodiments, the present invention provides a population of live attenuated recombinant picomaviruses comprising a library of exogenous (e.g., non-picornavirus) nucleic acid molecules, such that the population encodes a library of exogenous polypeptides which are individually synthesized as a component of a recombinant polyprotein precursor. The recombinant polyprotein precursor is proteolytically cleaved to release the exogenous polypeptide. In exemplary embodiments, recombinant picomavirus populations of the invention comprise a library of nucleic acid molecules that encode polypeptides from a pathogenic organism. Particularly, the invention provides populations of recombinant picomaviruses that comprise libraries of defined antigens from a particular pathogen or group of related pathogens.

Recombinant virus populations of the invention are useful in generating or enhancing an immune response to a pathogenic organism. Thus, the invention provides methods of eliciting an immune response to a pathogenic organism. The methods generally involve administering to an individual a population of recombinant viruses comprising a library of exogenous nucleic acid molecules. Member viruses of the population enter cells in the individual, where exogenous polypeptides are produced. Exogenous polypeptides are presented on the cell surface, or are secreted from the cell in which they are produced.

An advantage of the populations of recombinant virus populations, e.g., picornavirus populations, of the invention is that the parent viruses are live, attenuated viruses, and are thus safe for administration to humans and non-human animals.

In some aspects the invention provides methods of eliciting an immune response to an antigen, involving administering to an individual a first population of recombinant virus, e.g., a picornavirus, of a first strain, and, after a period of time, administering a second population of recombinant virus, e.g., a picornavirus, of a second strain. An immune response to the first population does not hinder replication of the second, or “booster,” population, since they are of different strains.

In some embodiments, the populations comprise defined antigens. In these embodiments, the antigens produced in cells after infection by a population of recombinant viruses (e.g., picornaviruses) are a selected subset of the total complement of proteins from a pathogen. Thus, rather than providing to an individual antigens that may be irrelevant in terms of immunoprotection, the individual is exposed only to those antigens that are most likely to provide immunoprotection against a pathogen.

### POPULATIONS OF RECOMBINANT REPLICATION-COMPETENT VIRUSES

The present invention provides a population of replication competent viruses comprising a plurality of member viruses. The replication-competent virus used in the population is any virus capable of replicating in the subject to which it is to be administered. In some embodiments, the replication-competent virus is a virus that naturally infects a eukaryotic cell of a mammalian host organism and which exhibits attenuated pathogenicity in that host. Suitable replication-competent viruses for use in the present invention include DNA viruses and RNA viruses, including, but not limited to, picornaviruses, flaviviruses, adeno-associated virus, adenovirus, lentiviruses, etc. A wide variety of such viruses are known in the art and are publicly available.

Each member virus of the population contains an inserted exogenous nucleic acid molecule which encodes a different exogenous polypeptide, peptide, protein or fragment thereof from the other member viruses in the population. The inserted exogenous nucleic acid molecule is operably linked to a sequence which facilitates expression of the exogenous nucleic acid molecule in the subject to which the viral population is to be administered. For example, the exogenous nucleic acid molecule can be inserted into a parent virus such that the encoded exogenous polypeptide is part of a recombinant polyprotein precursor. Alternatively, the exogenous nucleic acid molecule can be inserted into the parent virus such that the exogenous nucleic acid molecule is operably linked to a promoter such that the exogenous nucleic acid molecule is transcribed, and the exogenous protein translated. Another alternative is to link the exogenous nucleic acid molecule to an internal ribosome entry site (IRES) such as the encephalomyocarditis virus internal ribosome entry site (Ghattas, I. R. et al. (1991), Mol. Cell Biol., 11:5848-5859). Such viruses can be constructed using techniques well known in the art. For example, the exogenous nucleic acid molecule is inserted 3′ of an IRES in a retroviral vector.

In some embodiments, each member virus contains an inserted nucleic acid which encodes a polypeptide, peptide, protein, or fragment thereof which is derived from a pathogenic organism against which it is desired to induce an immune response. In further embodiments of the invention, the inserted nucleic acids included in the population may together comprise a substantial portion of the coding sequences from the genome of the pathogenic organism. For example, in some embodiments, the inserted nucleic acids included in the population may comprise at least 90%, at least 85%, at least 80%, at least 70%, at least 60%, at least 50%, or from about 10% to about 25%, from about 25% to about 50%, or from about 50% to about 90%, or more, of the coding sequences in the genome of the pathogenic organism.

Alternatively, in some embodiments, the inserted nucleic acids may span a substantial portion of the candidate coding sequences suspected of encoding antigenic peptides or polypeptides from the organism against which it is desired to induce an immune response. For example, the candidate coding sequences may encode proteins suspected of being expressed on the surface of the pathogenic organism or proteins suspected of being, or known to be, involved in the pathogenicity of the organism.

Viral populations of the invention are useful in inducing an immune response to an organism in which the antigens necessary to generate the desired level of immune response, such as a protective immune response, are not known. For example, in embodiments in which the inserted nucleic acid molecules encompass a substantial portion of the coding sequences from the genome of the organism against which it is desired to induce an immune response or a substantial portion of the coding sequences suspected of encoding antigenic peptides or polypeptides from the organism against which it is desired to induce an immune response, an immune response will be induced against one or more of the encoded peptides, polypeptides, proteins or portions thereof which harbor an antigenic site. In fact, since the replication-competent viruses will be present at significant levels in the subject to which they are administered, in some embodiments, an immune response may be induced against all or most of the encoded peptides, polypeptides, proteins or portions thereof which harbor an antigenic site.

In some embodiments, the invention provides a population of recombinant picornaviruses, particularly live attenuated recombinant picornaviruses, which comprise exogenous nucleotide sequences (i.e., nucleotide sequence from other than the parent picornavirus) which encode exogenous polypeptides (i.e., polypeptides from other than the parent picornavirus). A population of recombinant picornaviruses comprises a plurality of member viruses. Such populations of recombinant picornaviruses are useful in eliciting an immune response to the exogenous polypeptide.

A population of replication-competent recombinant viruses comprises at least a first member virus that comprises a first exogenous polypeptide coding sequence, and a second member virus that comprises a second exogenous polypeptide coding sequence, wherein the first polypeptide and the second polypeptide differ from one another in amino acid sequence by at least one amino acid. A member virus of a population of replication-competent recombinant viruses comprises an exogenous nucleic acid sequence that encodes an exogenous polypeptide that differs by at least one amino acid from all other member viruses.

A population of recombinant viruses generally comprises two, three, four, five, six, seven, eight, nine, ten, 11, 12, 13, 14, 15, 16, 17, 18, 19, 20, or more, members, up to about 30 members or more. Thus, for example, a population of recombinant viruses comprises a first and a second, at least a first, second and third, at least a first, second, third, and fourth, at least a first, second, third, fourth, and fifth, at least a first, second, third, fourth, fifth, and sixth, or additional, members, wherein each of these members comprises an exogenous nucleic acid molecule that encodes an exogenous polypeptide that differs from all the other members by at least one amino acid. In many embodiments, each member comprises an exogenous nucleic acid molecule that encodes an exogenous polypeptide that is antigenically distinguishable from all the other members, e.g., that comprises a different epitope from all the other members.

Each member may be present in the population in multiple copies. Furthermore, each member of the population need not be present in equal numbers. The number of copies of each member virus in the population can be any number consistent with the intended use of the population. In some embodiments, a population of recombinant picornaviruses is provided in which a member virus is present in the population in an amount of from about 50 to about 10⁵, from about 10² to about 10⁵, or from about 10³ to about 10⁴ or more, plaque forming units (PFU).

In some embodiments, the exogenous polypeptides encoded by two or more of the member viruses are overlapping. Thus, in certain embodiments, a population of recombinant picornaviruses comprises exogenous nucleic acid molecules that encode two or more overlapping polypeptides.

The length of the exogenous nucleic acid molecule encoding the exogenous polypeptide can be any length consistent with its intended use. For example, in some embodiments, the length of the exogenous nucleic acid molecule can range from about 12 nucleotides (nt) to about 1200 nt, from about 24 nt to about 900 nt, from about 30 nt to about 825 nt, from about 75 nt to about 750 nt, from about 90 nt to about 675 nt, from about 120 nt to about 600 nt, from about 150 nt to about 525 nt, from about 180 nt to about 450 nt, from about 210 nt to about 375 nt, or from about 240 nt to about 300 nt.

In some embodiments, each member virus in the population of replication-competent viruses contains a defined nucleic acid derived from an organism against which an immune response is to be induced. The defined nucleic acid encodes a defined peptide, polypeptide or protein or portion thereof (e.g., a portion comprising an antigenic determinant(s)) from the organism. The defined nucleic acid may be obtained using any of a variety of techniques familiar to those skilled in the art. For example, the defined nucleic acid may be obtained by performing an amplification reaction, such as a PCR reaction, using primers which hybridize to the sequences to be located at the ends of the defined nucleic acid. Alternatively, in some embodiments, the defined nucleic acids to be included in the member viruses may be obtained by performing a restriction digest on nucleic acids from the organism against which the immune response is to be induced, isolating the desired fragments, and inserting them into the viral vectors.

In some embodiments, a population of recombinant viruses of the invention comprises a subset (a “sublibrary”) of nucleic acid molecules encoding a subset or selected set of polypeptides (or a subset of overlapping polypeptides) from a given pathogen or group of related pathogens. The subset is selected from the total complement of proteins from the pathogen or group of related pathogens. Selection of nucleic acid molecules to be included in the subset can be made on the basis of known properties of particular polypeptides, including, e.g., immunogenicity of encoded polypeptides; the magnitude of an individual's immune response to an encoded polypeptide; the nature of an individual's immune response to an encoded polypeptide (e.g., whether an evoked immune response is a humoral immune response, a cellular immune response, a mucosal immune response, etc.); timing of synthesis of a polypeptide after infection by a pathogen (e.g., whether a polypeptide is produced “cearly” or “late” after infection of a cell); and the like.

Thus, for example, a library comprising the full complement of human immunodeficiency virus-1 (HIV-1) proteins may comprise a gag, pol, env, nef, tat, vif, vpr, vpu, vpx, and vpr polypeptide or immunogenic or antigenic portions thereof. A sublibrary of HIV-1 proteins may comprise two or more of the foregoing. For example, a subset of proteins from HIV-1 may comprise of gag, pol, env, and nef.

As one non-limiting example, a population of recombinant viruses comprises at least two members encoding at least two polypeptides comprising a human immunodeficiency virus (HIV) polypeptide selected from the group consisting of amino acids 2-128 of the HIV gag polypeptide, amino acids 117-248 of the HIV gag polypeptide, amino acids 233-364 of the HIV gag polypeptide, amino acids 362-509 of the HIV gag polypeptide, amino acids 29-146 of the HIV pol polypeptide, amino acids 218-330 of the HIV pol polypeptide, amino acids 290-472 of the HIV pol polypeptide, amino acids 397-530 of the HIV pol polypeptide, amino acids 490-631 of the HIV pol polypeptide, amino acids 597-767 of the HIV pol polypeptide, amino acids 828-981 of the HIV pol polypeptide, amino acids 18-164 of the HIV env polypeptide, amino acids 71-211 of the HIV env polypeptide, amino acids 148-249 of the HIV env polypeptide, amino acids 237-380 of the HIV env polypeptide, amino acids 335-498 of the HIV env polypeptide, amino acids 486-632 of the HIV env polypeptide, amino acids 526-698 of the HIV env polypeptide, amino acids 712-879 of the HIV env polypeptide, amino acids 1-145 of the HIV nef polypeptide, amino acids 126-262 of the HIV nef polypeptide, and amino acids 1-130 of the HIV tat polypeptide.

For pathogens that exist in multiple forms, e.g., multiple serotypes, a population can comprise multiple versions of the same polypeptide (i.e., the same polypeptide from two or more serovars), so that an immune response against more than one serotype is induced. As one non-limiting example, variants of influenza virus hemagglutinin and/or neuraminidase proteins are encoded by members of a population of recombinant picornaviruses. Those skilled in the art are aware of variant proteins of a variety of pathogens. See, e.g., Barbour and Restrepo (2000) Emerg. Infect. Dis. 6:449-457; and DeJong et al. (2000) J. Infect. 40:218-228.

Exogenous antigenic polypeptides encoded by member viruses can be any length consistent with their intended use. In some embodiments, exogenous polypeptides are from about 4 to about 6, from about 6 to about 10, from about 10 to about 15, from about 15 to about 20, from about 20 to about 25, from about 25 to about 50, from about 50 to about 75, from about 75 to about 100, from about 100 to about 125, from about 125 to about 150, from about 150 to about 175, from about 175 to about 200, from about 200 to about 225, from about 250 to about 300, from about 300 to about 350, or from about 350 to about 400 amino acids in length. For example, the exogenous antigenic polypeptides can be from about four to about 100, from about 100 to about 250, or from about 250 to about 400 amino acids in length.

A library of nucleic acid molecules or a subset of a library of nucleic acid molecules may encode overlapping polypeptides. Exogenous polypeptides can overlap by about 1, about 5, about 10, about 20, about 25, about 30, about 35, about 40, about 45, about 50, or about 60 amino acids or more, depending, e.g., on the length of the encoded polypeptide.

Exogenous polypeptides can be from any of a variety of pathogenic organisms, or from a plurality of pathogenic organisms. Exogenous polypeptides are from pathogenic organisms, including, but not limited to, Plasmodium spp.; Eimeria spp.; Schistosoma spp.; Trypanosoma spp.; Plasmodium spp.; Leishmania spp.; Cryptosporidia spp.; Toxoplasma spp.; Pneumocystis spp.; Vibrio cholerae; Streptococcus pyogenes; Neisseria meningitidis; Neisseria gonorrhoeae; Corynaebacterium diphtheriae; Clostridium tetani; Branhamella catarrhalis; Bordetella pertussis; Haemophilus spp. (e.g., influenzae); Chlamydia spp.; Enterotoxigenic Escherichia coli; Human Immunodeficiency virus, type I; Human Immunodeficiency virus, type II; Simian Immunodeficiency virus; Human T lymphotropic virus, type I and II; Respiratory syncytial virus; Hepatitis A virus; Hepatitis B virus; Hepatitis C virus; Herpes simplex virus, type I; Herpes simplex virus, type II; Cytomegalovirus; hifluenza virus; Parainfluenza virus; Poliovirus; Rotavirus; Coronavirus; Rubella virus; Measles virus; Mumps virus; Varicella; Epstein Barr virus; ebola virus; Adenovirus; Papilloma virus; Yellow Fever virus; Rabies virus; Candida spp. (especially albicans); Cryptococcus spp. (especially neoformans); Blastomyces spp. (dermatitidis); Histoplasma spp. (especially capsulatum); Coccidioides spp. (especially immitis); Paracoccidioides spp. (especially brasiliensis); and Aspergillus spp.

As shown in the Examples, a series of overlapping nucleic acid molecules from Simian Immunodeficiency Virus was generated. Table 1 provides the protein fragments encoded by the series of nucleic acid molecules. Thus, in some embodiments, the invention provides a population of recombinant viruses comprising a set of overlapping nucleic acid molecules encoding overlapping peptides from SIV, as shown in Table 1. In other embodiments, the invention provides a recombinant viral population comprising a set of nucleic acid molecules encoding an analogous set of exogenous peptides from other immunodeficiency viruses, including human immunodeficiency virus (HIV). Since the nucleotide sequences of various strains of HIV are publicly available, those skilled in the art can readily generate a set of exogenous nucleic acid molecules encoding such polypeptides from various strains of HIV.

Exogenous polypeptides can also be from tumor-associated antigens. Any of a variety of known tumor-associated antigens (TAA) can be inserted into picomavirus of the invention. The entire TAA may be, but need not be, inserted. Instead, a portion of a TAA, e.g., an epitope, particularly an epitope that is recognized by a CTL, may be inserted. Tumor-associated antigens (or epitope-containing fragments thereof) which may be inserted into picornavirus include, but are not limited to, MAGE-2, MAGE-3, MUC-1, MUC-2, HER-2, high molecular weight melanoma-associated antigen MAA, GD2, carcinoembryonic antigen (CEA), TAG-72, ovarian-associated antigens OV-TL3 and MOV18, TUAN, alpha-feto protein (AFP), OFP, CA-125, CA-50, CA-19-9, renal tumor-associated antigen G250, EGP-40 (also known as EpCAM), S100 (malignant melanoma-associated antigen), p53, prostate tumor-associated antigens (e.g., PSA and PSMA), and p21ras.

### Construction of Recombinant Picornaviruses

Replication-competent recombinant viruses comprising nucleic acids encoding exogenous polypeptides or portions thereof from a pathogenic organism may be generated using standard molecular biology techniques. In such techniques, the nucleic acids encoding the exogenous polypeptides or portions thereof are inserted into the replication-competent virus in a manner which facilitates the expression of the encoded polypeptide or portion thereof.

The replication-competent virus into which the nucleic acids encoding exogenous polypeptides are inserted may be a DNA virus, a RNA virus, or a retrovirus capable of replicating in a mammalian subject in which it is desired to induce an immune response. Replication-competent viruses suitable for use in the present invention include, but are not limited to, picornaviruses, flaviviruses, adeno-associated virus, adenovirus, lentiviruses.

Poliovirus is an example of a picornavirus. Poliovirus is a single-stranded positive-strand RNA virus. Viral particles are about 22-30 nm in size. The viral genome encodes a polyprotein precursor that is proteolytically processed. The parent wild-type viral genome can tolerate exogenous nucleic acid molecules up to about 800 bases in length. In the description below of recombinant picornaviruses of the invention, poliovirus is exemplified. The invention is not limited to poliovirus; rather, the following description is generally applicable to all replication-competent viruses, including picornaviruses. Picornaviruses useful in the present invention include, but are not limited to, replication-competent, attenuated strains of enteroviruses, poliovirus, FMDV, rhinovirus, echoviruses, and Hepatitis A virus.

Recombinant poliovirus can be constructed to include an exogenous nucleic acid sequence which encodes an exogenous polyprotein to be produced during the viral life cycle and a nucleic acid sequence which encodes an artificial proteolytic cleavage site for a viral or cellular protease which cleaves the precursor polyprotein produced by the parent virus. The two types of sequences can be present at any location in the parent virus genome, as long as their presence does not disrupt a viral sequence necessary for viral replication.

The exogenous nucleic acid sequence encoding an exogenous polypeptide can be incorporated into the genome of a virus at an end of the viral genome or at a site within the viral genome (i.e., at an internal site) and be produced during the viral life cycle as a precursor polyprotein which is cleaved by viral or cellular proteases which cleave the precursor polyprotein normally produced by the parent virus (i.e., the virus into which the exogenous nucleic acid sequence was introduced).

In one embodiment in which an exogenous nucleic acid sequence encoding an exogenous polypeptide is incorporated into the end of the viral genome, the order of sequences in the resulting recombinant viral genome is (from 5′ to 3′): 1) 5′ untranslated region of the parent virus; 2) unique start codon of the parent virus; 3) exogenous nucleic acid sequence; 4)nucleic acid sequence encoding an artificial proteolytic cleavage site; 5) second codon of the parent virus; and 6) remainder of the parent virus genome. The portion of the recombinant genome which is the 5′ untranslated region of the viral genome can be all or a portion of the 5′ untranslated region as it occurs in the parent virus.

In another embodiment in which an exogenous nucleic acid sequence encoding an exogenous polypeptide is incorporated within the viral genome, the order of sequences in the resulting recombinant viral genome is (from 5′ to 3′): 1) untranslated region of the parent virus; 2) unique start codon(s) of the parent virus; 3) the initial codon(s) of the translated region of the parent virus; 4) nucleic acid sequence encoding an artificial proteolytic cleavage site; 5) exogenous nucleic acid sequence; 6) nucleic acid sequence encoding an artificial proteolytic cleavage site; and 7) remainder of the parent virus genome.

The encoded exogenous polypeptide is expressed in the context of normal viral protein translation as a component of a recombinant or fusion precursor polypeptide (which includes the exogenous polypeptide, an artificial proteolytic recognition site or sites and the viral polyprotein). The recombinant precursor polypeptide is proteolytically processed by viral or cellular protease(s) which process the parent viral precursor polyprotein, resulting in release of the free exogenous protein from the viral proteins. The virus modified to include the exogenous sequences is referred to as the parent virus, which can be a native virus (either pathogenic or, preferably, non-pathogenic), an attenuated virus, a vaccine strain or a recombinant virus.

In some embodiments, replication-competent recombinant poliovirus which includes an exogenous nucleic acid sequence encoding an exogenous protein to be expressed and a nucleic acid sequence encoding a proteolytic cleavage site for the poliovirus 3C protease and/or 2A protease, incorporated into the end of the poliovirus genome or at a site within the poliovirus genome. It has been shown that the exogenous protein is expressed and freed from the poliovirus proteins by proteolytic processing. The resulting replication-competent recombinant polioviruses differ from the parent virus in that they include exogenous nucleic acid sequence(s) encoding an exogenous polypeptide or polypeptides and one or more artificial proteolytic cleavage sites and express the exogenous product during viral infection. The parent poliovirus can be a native or wild-type poliovirus, attenuated poliovirus, a vaccine strain or a recombinant or genetically engineered poliovirus (in which case the altered or mutated sequence does not encode an exogenous protein useful for the purposes described herein for the polioviruses which are the present invention).

In other embodiments, the exogenous nucleic acid sequence encoding the exogenous polypeptide and the nucleic acid sequence encoding the artificial proteolytic cleavage sites are positioned at an end of the polioviral genome, between the unique start codon and the second codon of the poliovirus genome such that the order of sequences in the recombinant genome is as follows (from 5′ to 3′): 1) 5′ untranslated region of the poliovirus genome; 2) poliovirus unique start (first) codon; 3) exogenous nucleic acid sequence; 4) artificial protease recognition site; 5) second codon of the poliovirus genome; 6) remainder of the poliovirus genome. As a result, expression of the recombinant polioviral genome produces a recombinant or fusion polyprotein precursor which includes the exogenous protein, the artificial protease cleavage site and the poliovirus polyprotein. Applicants have shown that proteolytic processing of the recombinant polyprotein precursor by the protease for which the artificial cleavage site is included results in production of the normal poliovirus protein components and freeing of the exogenous protein. Viral replication also ensues, but the exogenous protein is not included in the poliovirus virion.

There are a number of additional locations within the poliovirus genome at which the exogenous nucleic acid sequence encoding the exogenous polypeptide and the nucleic acid sequences encoding the artificial proteolytic cleavage sites can be positioned to produce replication-competent recombinant polioviruses that express the encoded product. These sites within the genome of the poliovirus include the junction between the Vp1 coding region and the 2A coding region, the junction between the 2A coding region and the 2B coding region and the junction between the 2C coding region and the 3A coding region. Polylinker/proteolytic processing motifs have been inserted at these sites and the resulting recombinant polioviruses have been shown to be replication-competent. Using the methods described herein and known methods, an exogenous nucleic acid sequence or sequences can be introduced at these sites. It will be appreciated that the exogenous nucleic acid sequence or sequences and proteolytic cleavage site(s) can be inserted at any position in the parent viral genome that allows the exogenous polypeptide to be liberated from the recombinant viral polyprotein precursor by proteolytic cleavage.

To facilitate processing of exogenous sequences within the interior of the viral polyprotein, it is necessary to include the appropriate proteolytic processing signals at both ends of the insert. Therefore, the order of sequences in the polioviral genome in which the exogenous nucleic acid sequence encoding the exogenous polypeptide and the nucleic acid sequence encoding the artificial proteolytic cleavage sites are, for example, inserted at the junction between the Vp1 coding region and the 2A coding region, is as follows (from 5′ to 3′): 1) 5′ untranslated region of the poliovirus genome; 2) poliovirus unique start codon; 3) Vp0 coding region; 4) Vp3 coding region; 5) Vp1 coding region; 6) nucleic acid sequence encoding artificial 3C protease recognition site or 2A protease recognition site; 7) exogenous nucleic acid sequence; 8) nucleic acid sequence encoding artificial 3C protease recognition site or 2A protease recognition site; 9) remainder of the poliovirus genome.

Additional features may be incorporated into the design of replication-competent recombinant viruses, such as polylinker sequences (e.g., EcoR1, NotI, BssH2, and XhoI) to facilitate the ease of insertion of desired foreign sequences into the recombinant vector. Also, variants, such as a poly-glycine tract, may be inserted adjacent to the inserted sequence so as to enhance the structural flexibility of the region and potentially increase the efficiency of proteolytic processing.

In some embodiments, more than one nucleic acid sequence encoding an exogenous protein or polypeptide to be produced is included in the recombinant replication-competent virus which, as a result, produces the corresponding number of proteins or polypeptides. The two or more nucleic acid sequences can each encode a different product or can encode the same product (e.g., if enhanced production of a protein or polypeptide is desired). Further, for poliovirus, the proteolytic cleavage site(s) can be the 3C cleavage site, the 2A cleavage site or both.

Although the present invention is exemplified by production of recombinant poliovirus, any virus in which proteolytic processing of a viral precursor protein occurs can be modified to produce recombinant virus which expresses an exogenous protein and processes it appropriately. For example, recombinant picornaviruses (e.g., enteroviruses, poliovirus, FMDV, rhinovirus, echoviruses, Hepatitis A virus) can be produced and used in a similar manner to that described for recombinant poliovirus. Similarly, any other replication-competent virus may be engineered to express an exogenous polypeptide or antigenic portion thereof.

In addition, the population can comprise two or more strains of virus. As one non-limiting example, Sabin-1 and Sabin-2 live attenuated strains of poliovirus can be used as the parent viruses. A library or sublibrary can be made in each of the Sabin-1 and Sabin-2 strains. The first and second strains can be administered as a combined population. Alternatively, the different strains can be used to generate separate populations of recombinant virus. Use of two different strain populations allows use of a population made in a first strain to increase an immune response to encoded polypeptides; and the population made in the second strain as a booster. The advantage of using two different strains for the initial vaccine and the booster vaccine is that the first strain will not impede replication of the second strain in the individual. In some embodiments, the booster (e.g., the second population) is administered to the individual about 12 hours, about 24 hours, about 2 days, about 4 days, about 1 week, about 2 weeks, about 1 month, about 2 months, about 3 months, about 4 months, about 6 months, about 1 year, about 2 years, or more, after the initial population.

In some embodiments, the replication-competent virus may be a virus which induces a mucosal immune response against the exogenous polypeptide or portion thereof. For example, the replication-competent virus may be a poliovirus. For example, effective induction of mucosal immunity would be advantageous in preventing or lessening infection by HIV, rotavirus, respiratory syncytial virus (RSV), Hepatitis A virus, poliovirus, papilloma virus, measles virus and the influenza viruses. The recombinant viruses are also useful for inducing immunity against bacterial diseases, such as those caused by Vibrio cholerae and enterotoxigenic E. coli. In addition, the recombinant poliovirus can be used to provide protection against infection by more than one organism by introducing more than one exogenous nucleic acid sequence (i.e., two or more nucleic acid sequences, each of which encodes an antigen from a different organism) into the genome of the parent poliovirus.

The gene sequences encoding the exogenous protein or portion thereof to be expressed by the recombinant virus according to the present invention, can be obtained by techniques known in the art, including but not limited to, chemical or enzymatic synthesis, purification from genomic DNA of the microorganism, by purification or isolation from a cDNA encoding a TAA, by cDNA synthesis from RNA of the microorganism, or by recombinant DNA methods (Maniatis et al., Molecular Cloning, A Laboratory Manual, 1982, Cold Spring Harbor Laboratory, Cold Spring Harbor, N.Y.).

### ROUTES OF ADMINISTRATION, FORMULATION, AND DOSAGES

The invention provides immunogenic compositions comprising a replication-competent recombinant virus population of the invention. When they are used to induce or enhance an immune response, the populations of recombinant viruses of the present invention are administered to an individual using known methods. They will generally be administered by the same routes by which conventional (presently-available) vaccines are administered and/or by routes which mimic the route by which infection by the pathogen of interest occurs. They can be administered in a composition which includes, in addition to the replication-competent recombinant virus, a physiologically acceptable carrier. The composition may also include an immunostimulating agent or adjuvant, flavoring agent, or stabilizer.

Conventional and pharmaceutically acceptable routes of administration include intranasal, intramuscular, intratracheal, intratumoral, subcutaneous, intradermal, vaginal, intrapulmonary, intravenous, rectal, nasal, oral and other parenteral routes of administration. Routes of administration may be combined, if desired, or adjusted depending upon the antigenic peptide or the disease. The immunogenic composition can be administered in a single dose or in multiple doses, and may encompass administration of booster doses, to elicit and/or maintain immunity.

The immunogenic compositions is administered in an “effective amount” that is, an amount of recombinant virus population that is effective in a selected route of administration to elicit or induce an immune response. An immune response is elicited to exogenous polypeptides encoded by member viruses and therefore to antigens produced by a pathogenic organism. In some embodiments, the amount of recombinant virus population is effective to facilitate protection of the host against infection, and/or to reduce a symptom associated with infection, by a pathogenic organism. In some embodiments, an “effective amount” of an immunogenic composition is an amount of recombinant virus population that is effective in a route of administration to elicit an immune response effective to reduce or inhibit tumor cell growth, to reduce tumor cell mass or tumor cell numbers, or to reduce the likelihood that a tumor will form.

The amount of recombinant virus population in each vaccine dose is selected as an amount which induces a desired immune response (including, but not limited to, an immunoprotective or other immunotherapeutic response) without significant, adverse side effects. Such amount will vary depending upon which specific immunogen is employed, whether or not the vaccine formulation comprises an adjuvant, and a variety of host-dependent factors. In some embodiments, each dose of immunogenic composition will be sufficient to generate, upon infection of host cells, about 1-1000 μg of exogenous protein, generally from about 1-200 μg, normally from about 10-100 μg exogenous protein. In some embodiments, about 1-1000 μg of recombinant virus population nucleic acid is administered.

Alternatively, in some embodiments, an effective dose of immunogenic composition is in a range of from about 10² to about 10⁷, from about 10³ to about 10⁶, or from about 10⁴ to about 10⁵ plaque forming units (PFU). An optimal amount for a particular vaccine can be ascertained by standard studies involving observation of antibody titers and other responses in subjects. The levels of immunity provided by the immunogenic composition can be monitored to determine the need, if any, for boosters. Following an assessment of antibody titers in the serum, optional booster immunizations may be desired. The immune response to the protein of this invention is enhanced by the use of adjuvant and or an immunostimulant.

In some embodiments, the compositions comprising the viral population may be administered using conventional devices including but not limited to syringes, devices for intranasal administration of compositions, gene guns, and vaccine guns. Thus, one embodiment of the present invention is a device comprising a member which receives the viral population (or composition comprising the viral population) in communication with a mechanism for delivering the composition to the subject.

### COMPOSITIONS COMPRISING RECOMBINANT VIRUS POPULATIONS OF THE INVENTION

The present invention further provides compositions, including pharmaceutical compositions, and immunogenic compositions, comprising a recombinant virus population of the invention.

Compositions comprising a recombinant virus population of the invention may include a buffer, which is selected according to the desired use of the recombinant virus population, and may also include other substances appropriate to the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995) Mack Publishing Co.

Pharmaceutical compositions can be prepared in various forms, such as granules, tablets, pills, suppositories, capsules, suspensions, sprays, suppositories, transdermal applications (e.g., patches, etc.), salves, lotions and the like. Pharmaceutical grade organic or inorganic carriers and/or diluents suitable for oral and topical use can be used to make up compositions containing the therapeutically active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

When used as an immunogenic composition, a recombinant virus population of the invention can be formulated in a variety of ways. In general, an immunogenic composition of the invention is formulated according to methods well known in the art using suitable pharmaceutical carrier(s) and/or vehicle(s). A suitable vehicle is sterile saline. Other aqueous and non-aqueous isotonic sterile injection solutions and aqueous and non-aqueous sterile suspensions known to be pharmaceutically acceptable carriers and well known to those of skill in the art may be employed for this purpose.

Optionally, an immunogenic composition of the invention may be formulated to contain other components, including, e.g., adjuvants, stabilizers, pH adjusters, preservatives and the like. Such components are well known to those of skill in the vaccine art. Adjuvants include, but are not limited to, aluminum salt adjuvants (Nicklas (1992) Res. Immunol. 143:489-493); saponin adjuvants; Ribi's adjuvants (Ribi ImmunoChem Research Inc., Hamilton, Mont.); Montanide ISA adjuvants (Seppic, Paris, France); Hunter's TiterMax adjuvants (CytRx Corp., Norcross, Ga.); Gerbu adjuvants (Gerbu Biotechnik GmbH, Gaiberg, Germany); and nitrocellulose (Nilsson and Larsson (1992) Res. Immunol. 143:553-557). In addition, other components that may modulate an immune response may be included in the formulation, including, but not limited to, cytokines, such as interleukins; colony-stimulating factors (e.g., GM-CSF, CSF, and the like); and tumor necrosis factor.

### USES OF RECOMBINANT VIRUS POPULATIONS OF THE INVENTION

The present invention provides methods for eliciting an immune response to an antigen(s), comprising administering a recombinant virus population of the invention to a mammalian subject, wherein the a member virus enters a cell, the exogenous polypeptide is synthesized by the host cell, and an immune response is elicited to the exogenous polypeptide.

In exemplary embodiments, the exogenous polypeptide is an antigenic polypeptide of a microbial pathogen. In these embodiments, recombinant virus populations are administered to a host to prevent, reduce, inhibit, or treat infection by the pathogen, or to prevent, reduce, inhibit, or treat symptoms of such pathogenic infection. Of particular interest is the prevention, reduction, inhibition, or treatment of infection or disease caused by microbial pathogens that, during the course of infection, are present intracellularly, e.g., viruses (e.g., HIV), bacteria (e.g. Shigella, Listeria, Streptococcus, Salmonella, and the like), parasites (e.g., malarial parasites (e.g., Plasmodium falciparum), trypanosomes, and the like), etc., e.g., as noted above. Antigenic polypeptides of such microbial pathogens are well known in the art, and can be readily selected for use in the present immunogenic compositions by the ordinarily skilled artisan.

In addition, a recombinant virus population of the invention can be used as a delivery vehicle to delivery any antigen to an individual, to provoke an immune response to the antigen. In some embodiments, recombinant virus population of the invention are used as bivalent or multivalent vaccine to treat human or veterinary diseases caused by infectious pathogens, particularly viruses, bacteria, and parasites. Examples of epitopes which could be delivered to a host in a multivalent recombinant virus population (e.g., in an immunogenic composition) of the invention include multiple epitopes from various serotypes of Group B streptococcus, influenza virus, rotavirus, and other pathogenic organisms known to exist in nature in multiple forms or serotypes; epitopes from two or more different pathogenic organisms; and the like.

Whether an immune response has been elicited to a pathogenic organism can be determined (quantitatively, e.g., by measuring a parameter, or qualitatively, e.g., by assessing the severity of a symptom, or by detecting the presence of a particular parameter) using known methods. Methods of measuring an immune response are well known in the art and include enzyme-linked immunosorbent assay (ELISA) for detecting and/or measuring antibody specific to a given pathogenic organism; and in vitro assays to measure a cellular immune response (e.g., a CTL assay using labeled, inactivated cells expressing the epitope on their cell surface with MHC Class I molecules). A biological sample obtained from the individual is used to test for the presence and/or quantity of antigen-specific antibody (e.g., serum IgG, mucosal IgA, etc.); and/or antigen-specific CTL. Suitable biological samples include, but are not limited to, serum; vaginal samples (e.g., fluids, cells); rectal samples (e.g., fluids, cells, etc.); blood; and the like. Whether a mucosal immune response is elicited can be determined using any known method, including, e.g., measuring secretory IgA, specific for an epitope(s) associated with the pathogenic organism, produced in a mucosal tissue.

In many embodiments, an immune response is elicited to each of the antigenic exogenous polypeptides encoded by the population of recombinant viruses. In other embodiments, an immune response is elicited to at least 90%, at least 80%, at least 70%, at least 60%, or at least 50% of the exogenous antigenic polypeptides.

Whether an immune response is effective to facilitate protection of the host against infection, or reduce symptoms associated with infection, by a pathogenic organism can be readily determined by those skilled in the art using standard assays, e.g., determining the number of pathogenic organisms in a host (e.g., measuring viral load, and the like); measuring a symptom caused by the presence of the pathogenic organism in the host (e.g., elevated body temperature; lower than normal CD4⁺ T cell counts; weight loss; secondary infections; and the like).

In some embodiments, a polypeptide antigen expressed on a given tumor cell (e.g., a tumor associated antigen; “TAA”) is inserted into a recombinant virus population of the invention as described herein. Such recombinant virus populations can be administered to an individual having, or suspected of having, a tumor. In some cases, such recombinant virus populations can be administered to an individual who does not have a tumor, but in whom protective immunity is desired. As is often the case, the immune system does not mount an immune response effective to inhibit or suppress tumor growth, or eliminate a tumor altogether. Tumor-associated antigens are often poorly immunogenic; perhaps due to an active and ongoing immunosuppression against them. Furthermore, cancer patients tend to be immunosuppressed, and only respond to certain T-dependent antigens. In these cases, introduction into the host of a recombinant virus population of the invention which expresses an exogenous peptide corresponding to an antigen expressed on the tumor cell surface can elicit an immune response to the tumor in the host.

A recombinant virus population comprising a TAA can be administered to an individual as described above. Whether an immune response is elicited to a given tumor can be determined by methods standard in the art, including, but not limited to, assaying for the presence and/or amount of TAA-specific antibody in a biological sample obtained from the individual, e.g., by enzyme-linked immunosorbent assay (ELISA), radioimmunoassay (RIA), and the like; assaying for the presence and/or numbers of CTLs specific for a TAA; and the like. Examples of how to assay for the presence and/or numbers of antigen-specific CTLs are found in the Examples section herein below. Standard immunological protocols may be used, which can be found in a variety of texts, including, e.g., Current Protocols in Immunology (J. E. Coligan, A. M. Kruisbeek, D. H. Margulies, E. M. Shevach and W. Strober Eds. 1991).

Whether an immune response is effective in reducing the number of tumor cells in an individual can be determined by standard assays, including, but not limited to, measuring tumor cell mass, measuring numbers of tumor cells in an individual, and measuring tumor cell metastasis. Such assays are described in the Examples section herein below.

Using the methods and compositions described herein in connection with the subject invention, an immunoprotective response against microbial infection can be induced in any subject, human or non-human, susceptible to infection by a microbial pathogen. Where the recombinant virus population comprises an exogenous nucleic acid sequence encoding a TAA, the subject may be one that is known to have cancer, is suspected of having cancer, or does not have cancer, but in whom immunity to cancer is to be induced.

The recombinant viral populations are also useful for identifying nucleic acids which encode a peptide, polypeptide, protein or portion thereof which harbors an antigenic determinant. In such embodiments, a plurality of defined nucleic acid inserts are cloned into replication-competent viral vectors such that the peptides, polypeptides, proteins, or portions thereof will be expressed by the viral vectors as described above. For example, in some embodiments, the nucleic acid inserts may span substantially all of the coding regions of the organism against which it is desired to induce an immune response or a substantial portion of the candidate coding sequences suspected of encoding antigenic peptides or polypeptides from the organism against which it is desired to induce an immune response.

The viral population is administered to a subject in sufficient quantity to induce an immune response. After a sufficient period of time for an immune response to be induced has elapsed, serum is obtained from the subject. The serum is applied to a support, such as a nylon or nitrocellulose membrane or wells in a microtiter plate, which has the peptides, polypeptides, proteins or portions thereof which are encoded by the inserted nucleic acids bound thereto in a unique location under conditions which allow antibodies directed against the peptides, polypeptides, proteins, or portions thereof to specifically bind to their targets.

After rinsing away non-specifically bound antibodies, antibodies specifically bound to the peptides, polypeptides, proteins, or portions thereof are then detected to determine which of the peptides, polypeptides, proteins or portions thereof induced an immune response. For example, the antibodies may be detected using secondary antibodies having a detectable moiety, such as a radioactive isotope or enzyme which generates a detectable product, bound thereto. For example the peptides, proteins, polypeptides or portions thereof which induce an immune response may be detected by performing a Western blot or ELISA analysis.

The peptides, proteins, polypeptides or portions thereof identified as described above may then be used in a composition for inducing an immune response. The composition may comprise the purified peptides, proteins, polypeptides or portions thereof themselves or, alternatively, the composition may comprise a population of replication competent viruses, such as those described above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Standard abbreviations are used, e.g., hr, hour; min, minutes; sec, seconds; and the like. Unless indicated otherwise, parts are parts by weight, molecular weight is weight average molecular weight, temperature is in degrees Celsius, and pressure is at or near atmospheric.

### Example 1

### Protection From SIV Vaginal Challenge Using Sabin Poliovirus Vectors

### MATERIALS AND METHODS

### Plasmnids

The Sabin 1 plasmid (which we call pS1 for simplicity), was kindly provided by A. Nomoto (construct pVS(1)IC-0(25). Lehner et al.(1996) Nat. Med. 2:767-775; Omata et al. (1984) Gene 32:1-10. The entire Sabin 1 CDNA in pS1 was sequenced in our laboratory by the fluorescent dye terminator method using an ABI 310 machine (Perkin Elmer, Calif.). The accuracy of the genome sequence as published was confirmed. Nomoto et al. (1982) Proc. Natl. Acad. Sci. USA 79:5793-5797. To construct pSabRV1, first the EcoRI and XhoI sites upstream of the T7 promoter of pS1 were eliminated by inserting a SalI linker (oligos C and D) at that position to create plasmid pS1XT. Then the 747 bp BstEII fragment of pMoV2.11—containing the duplicated 2A^{Pro} cleavage site, the 5 glycine spacer, and the EcoRI, NotI, and XhoI cloning sites—was swapped into pS1XT to create pSabRV1. Accuracy of the pSabRV1 construct was confirmed by restriction digest and DNA sequencing. This DNA swap between pMoV2.11 and Sabin 1 results in Sabin 1 gaining three wild type (wt) coding changes in 2A and one in 2B. None of the changes are associated with neurovirulence or other wt phenotypes. All pSabRV1 plasmids contain an AmP^{R} selectable marker.

Plasmid pS1, pS1XT, and pSabRV1 were grown by electroporation into SURE cells (Stratagene, Calif.) and plated on LB+ampicillin agar plates for 20-24 hours at 37° C. Single colonies were then inoculated into 50 ml cultures of LB+ampicillin (50 μg/ml) and grown at 30° C. for 16-18 hrs. Note: growth conditions for the Sabin 1 derivative plasmids (pS1, pS1XT, and pSabRV1)are important, as rearrangements of the plasmids and very low plasmid yields are frequently seen otherwise. Plasmid DNA was isolated from cells by the QiaFilter Midiprep technique (Qiagen, Calif.).

For pSabRV1-SIV clones, SlVmac239 plasmids p239SpSp5′, p239SpE3′, and pSIV239opennef (obtained from the AIDS Research and Reference Reagent Program, courtesy of Ronald Desrosiers were used as the PCR template to generate SIV inserts. Kestler et al. (1990) Science 248:1109-1112. Inserts were amplified using Pfu Turbo high-fidelity DNA polymerase, using conditions recommended by the manufacturer (Stratagene, Calif.). A complete table of the 40 oligos used for these reactions is available upon request. PCR fragments were purified on Qiaquick spin columns, digested with DpnI restriction enzyme (to eliminate any input SIV plasmid carried over), EcoRI, and XhoI, and then Qiaquick spin column purified a second time.

Vector pSabRV1 plasmid was cut with EcoRI and XhoI, Qiaquick spin column purified, and then quantified by agarose gel electrophoresis. Gel purification of vector was generally avoided. SIV inserts were ligated into pSabRV1 using NEB T4 DNA ligase (New England BioLabs, MA) in an overnight reaction at 16° C. containing 25 ng pSabRV1 and 20 ng SIV insert DNA. Ligations were dialyzed on 13 mm 0.0251 μm VSWP membranes (Millipore, Mass.) against 50 ml of deionized H₂O for 10 minutes. Then 1 μl of ligation was electroporated into 25 μl SURE cells in a 0.1 cm cuvette (BTX ElectroCell 600 electroporator conditions: 129 ohm , 1,400 V, 5 msec pulse). One ml of LB was immediately added to the cuvette, and 20-200 μl of electroporated SURE cells were plated onto LB+ampicillin plates and incubated at 37° C. overnight. Further culturing and DNA isolation was as described above. All plasmid clones were analyzed by restriction digest and all inserts were DNA sequenced in their entirety to confirm that the appropriate clone had been obtained and was not mutated.

Sabin 2 early passage virus (SO+3) was kindly provided by K. Chumakov. HeLa cells were infected with Sabin 2 at an MOI of>1 and incubated at 37° C. Cells were harvested at 9 hrs post-infection, RNA was extracted using RNeasy (Qiagen, Santa Clarita, Calif.), and cDNA was synthesized using random primed Superscript II (Life Technologies, Gaithersburg, Md.). Full length Sabin 2 was PCR amplified with primers SAB21 and SAB24, using XL polymerase (Perkin Elmer, Calif.), 2 mM Mg(OAc), and 500 μM dNTPs, with conditions: 3) min at 94° C., 8 minutes at 65 ° C., for 30 cycles. The full length Sabin 2 genome was then Qiaquick spin column purified, digested with SalI and HindIII, and ligated into SalI/HindIII digested pUC18. Ligations were introduced into DH5α chemically competent cells as recommended by the manufacturer (Life Technologies, MD). Plasmid minipreps of clones were analyzed by restriction digest and tested for the ability to produce infectious virus.

The three plasmid clones that produced virus (pS2-2, pS2-3, and pS2-10) were sequenced and the genome sequence was compared to the Sabin 2 consensus sequence generated by Pollard et al. ((1989) J. Virol. 63:4949-4951). Two coding mutations in pS2-10 were identified, one in Vp2 and one in 3C. The latter was corrected by swapping the 374 bp BsiWI-NcoI DNA fragment from a clone (pS2-3) with no 3C mutation into pS2-10 to create pS2-10F. We have since fixed the other coding mutation in pSabRV2 (nucleotide 1492, a.a. 249, F to L in Vp2) by site-directed mutagenesis and checked the resulting pSabRV2.2 by DNA sequencing. Viruses derived from pS2-10, pS2-10F, pSabRV2, and pSabRV2.2 all grow identically to Sabin 2 by plaque assay.

To generate pSabRV2, the 60 bp cloning site—which contains a 5 glycine spacer and AvrII and NotI restriction sites flanked by 2A^{Pro} cleavage sites (only the 5′ (or N terminal) cleavage site is counted in the 60 bp, since it contains modified codon usage (Tang et al. (1997) J. Virol. 71:7841-7850) and the 3′ (or C terminal) cleavage site is endogenous and essential)—was cloned into pS2-10F. A BstEII-SnaBI fragment containing the unique SabRV2 cloning sites was generated by overlapping PCR of DNA fragments B and S, and digestion with BstEII and SnaBI. DNA fragment B was made by PCR (Pfu Turbo, Stratagene, Calif.) using oligos B1 and oligo B2. Similarly, DNA fragment S was generated by PCR using oligos S1(63 nt long, 45 nt of which overlap with oligo B2, which together contain the full pSabRV2 cloning site) and S2. Both PCR fragments were gel purified using Qiagen and used together as template with oligos B1 and S2 in an overlapping PCR reaction to generate a 1635 bp fragment containing the 60 bp SabRV2 cloning site flanked by the BstEII and SnaBI restriction sites. The digested BstEII-SnaBI fragment was ligated into BstEII/SnaBI digested pS2-10F to create pSabRV2. Viruses derived from pS2-10, pS2-10F, pSabRV2, and pSabRV2.2 all grow identically to Sabin 2 by plaque assay).

For pSabRV2-SIV cloning, SIV PCR fragments were generated as described above (using similar oligos; a complete list of all 42 oligos is available upon request) and cloning was done comparably to that of pSabRV1-SIV's, except AvrII/NotI digestions were used and Xl1-Blue cells (Stratagene, Calif.) were used for transformations. Stocks of pSabRV2-SIV plasmids were made by inoculating single colonies of transformed XL1-Blue cells (grown overnight on LB+amp plates) into 5 ml cultures of LB+ampicillin (50 μg/ml) and grown at 37° C. for 8-14 hrs. Plasmid DNA was isolated from cells by the Qiafilter Miniprep technique (Qiagen, Calif.). All clones were analyzed by restriction digest and all inserts were DNA sequenced in their entirety to confirm that the appropriate clone had been obtained and was not mutated.

All vectors and plasmids are readily available to any interested investigator.

### Oligonucleotides

The nucleotide sequences of oligonucleotides used in these studies are given as 5′ to 3′.

| | | |
|---|---|---|
| A = | GGTGGGGGAGGTGAATTCATGGTGAGCAAGGGCGAGGAG | (SEQ ID NO:01) |
| E = | GTGGTCAGATCCTCGAGCTTGTACAGCTCGTCCATGCCG | (SEQ ID NO:02) |
| C = | AATTGGTTCCTGGTCGACCGATGATCCGCG | (SEQ ID NO:03) |
| D = | TCGACGCGGATCATCGGTCGACCAGGAACC | (SEQ ID NO:04) |
| B1 = | ACATATTCGAGATTTGAC | (SEQ ID NO:05) |
| B2 = | TGCGGCCGCTGCCCTAGGCCCTCCGCCACCTCCATGAC | (SEQ ID NO:06) |
| | CGAAACCGTATGTGGTCAGACCCTTTTCTGG | |
| S1 = | GGTTTCGGTCATGGAGGTGGCGGAGGGCCTAGGGCAGCG | (SEQ ID NO:07) |
| | GCCGCAGGATTAACGACTTATGGA | |
| SAB21 = | AAAAGGTCGACTAATACGACTCACTATAGGTTAAAACAGCT | (SEQ ID NO:08) |
| | CTGGGGTTG | |
| SAB24 = | GGGGGAAGCTTAGGCCTTTTTTTTTTTTTTTTTTTTCCTCCGAATT | (SEQ ID NO:09) |
| | AAAGAAAAAT | |
| S1-3580R = | GCCCTGGGCTCTTGATTCTGT | (SEQ ID NO:10) |
| S2-3151F = | GAAGGCGATTCGTTGTAC | (SEQ ID NO:11) |
| S2-3518R = | CTTGATTCAGCCACTAAG | (SEQ ID NO:12) |

### Transcriptions and Electroporations

Transcriptions were generally done using T7 RNA Polymerase (150 U) from New England Biolabs, using the supplied transcription buffer supplemented with 40 U RNAsin (Promega, Wis.), and 1.25 mM NTPs. Plasmid templates (1-3 μg) were first linearized with ClaI (pS1, or pSabRV1 vector) or HinDIII (pS2-10F or pSabRV2) for 1 hr at 37° C. in a 20 μl volume. The restriction enzyme was then inactivated for 10 min at 65° C. Once linearized, plasmid template was added to the full transcription mixture (total volume 200 μl), and transcription was allowed to proceed for 60-90 min at 37° C. before terminating the reaction by freezing at −80° C. RNA quality and quantity was assessed by agarose gel electrophoresis before use in subsequent experiments. RNA from transcription reactions was used directly, without purification, in electroporations.

Electroporations were done using HeLa S3 cells at 40-75% confluence, plated the previous day, which were then trypsinized, centrifuged, and resuspended at a concentration of 3×10⁶ cells/ml in Ca⁺⁺/Mg⁺⁺—free phosphate buffered saline (on some occasions, 293 cells were used in an identical manner). 800 μl of cells was added to a cold 0.4 cm electroporation cuvette (BioRad, CA; or BTX, CA), 10-40 μg RNA was added to cells, cuvette was flicked multiple times to resuspended cells that had settled, and cuvette was immediately electroporated in a BTX electroporator with settings: 950 μF, 24 ohm, and 300 V. The entire contents of the cuvette was then added to a 6 cm dish (10 cm dishes were used for SabRV2 viruses) with 3 ml of warm DMEM/F12+10% FCS (see (23) for related details). These electroporation conditions consistently give a 50-80% electroporation efficiency, resulting in first generation (P₀) virus stocks.

Sabin1 and SabRV1 recombinants were grown at 32° C., as Sabin 1 has a tendency to acquire wild type characteristics when passaged multiple times at greater than 34° C. Rezapkin et al. (1998) Virol. 245:183-187. Sabin 2 (S2-10F), and SabRV2 recombinants were grown at 37° C. Plates were left until complete cytopathic effect (CPE) was observed; frequently 24-36 hrs for SabRV1 and SabRV2 recombinants.

HeLa S3 cells obtained from ATCC (ATCC stock+5−30 passages) were grown in DMEM/F12 medium (Gibco/Life Technologies) supplemented with 10% fetal calf serum (FCS) (Gibco/Life Technologies), penicillin/streptomycin, and L-glutamine. Adherent cell cultures were maintained at 10-80% confluence at 37° C.+6% CO₂. 293 cells were grown under the same conditions, but were sometimes left to 100% confluence.

### Viral Stocks, Passages, and Plaque Assays

P₀ viral stock were harvested from electroporated cells exhibiting full CPE by taking the cells and supernatant, and freeze/thawing 3 times with a dry ice/ethanol bath and a 37° C. water bath. Cellular debris was then pelleted by a 5 min, 300 g centriffigation, and P₀ viral stock supernatant was transferred to a fresh tube. Note: some of the MoV2.11, SabRV1, and SabRV2 recombinant viral stocks appeared to lose some titer upon multiple freeze/thaw cycles. This was not observed with normal wild type poliovirus. Therefore viral stocks were stored in constant temperature −30° C. or −80° C. freezers.

Concentration of several viruses was done using Centriprep concentration filters units with a molecular weight limit of 50kD (Milipore, Mass.). 12-15 ml of low-titer SabRV1-SIV or SabRV2-SIV viral stocks were spun in Centriprep filter units for 30 minutes at 3,000×g. This resulted in a 5-15 fold concentration of virus. Concentrated stocks were then titered by plaque assay.

Nine P₀ SabRV2-SIV viruses were mixed in equal amounts and passaged five times at an MOI of 0.1, at both 32° C. and 37° C. Identical data were obtained for passages at 32° C. and at 37° C. Passaging of SabRV2-SIV viruses was done by infecting 3×10⁶ HeLa cells in 10 cm plates at an MOI of 0.1 with the P₀ viral cocktail stock. Cells were incubated in 3 ml DMEM/F12 medium supplemented with 10% fetal calf serum at 32C. or 37° C.; and P₁ viral stock was harvested when complete CPE was observed (24-36 hrs post-infection). The same process was followed when carrying out passages P₂ through P₅. Each passage at MOI of 0.1 represents approximately 2 generations of viral replication. In total, P₅ viruses had gone through 10-12 generations of viral replication. The cocktail passages were tested for the presence of the SIV inserts by RT-PCR using primers in the poliovirus sequence flanking all of the SIV inserts.

All plaque assays were done as previously described. Crotty et al. (1999) J. Virol. 73:9485-9495; and Crotty et al. (2000) Nat. Med. 6:1375-1379. Plaque assays involving SabRV1 recombinants were incubated at 32° C. for 5 days post-infection, plaque assays involving SabRV2 recombinants were incubated at 37° C. for 4 days post-infection.

Viruses used in the SabRV1-SIV and SabRV2-SIV vaccines are listed in Table 1.

TABLE 1
SabRV1/2-SIV vaccine library cocktails

| Sabin1 virus name | Sabin2 virus name | Amino acid coverage |
|---|---|---|
| SabRV1-Gag1 | SabRV2-Gag1 | Gag 2-134 (p17) |
| SabRV1-Gag2 | SabRV2-Gag2 | Gag 92-263 (p17/p24) |
| | SabRV2-Gag3N | Gag 133-299 |
| | SabRV2-Gag3C | Gag 266-432 |
| SabRV1-Gag4 | SabRV2-Gag4 | Gag 362-509 (p24/p9) |
| SabRV1-Pol6 | | Pol (-)29-146 (protease) |
| SabRV1-Pol7 | SabRV2-Pol7 | Pol 97-266 |
| SabRV1-Pol8 | SabRV2-Pol8 | Pol 218-330 |
| SabRV1-Pol9 | SabRV2-Pol9 | Pol 290-472 |
| SabRV1-Pol10 | SabRV2-Pol10 | Pol 397-530 |
| SabRV1-Pol11 | SabRV2-Pol11 | Pol 490-631 |
| SabRV1-Pol12 | | Pol 597-767 |
| SabRV1-Pol14 | SabRV2-Pol14 | Pol 828-981 |
| SabRV1-Env15 | | Env 18-164 (gp120) |
| | SabRV2-Env15C | Env 71-211 (gp120) |
| SabRV1-Env16M | SabRV2-Env16M | Env 148-249 (gp120) |
| SabRV1-Env17 | SabRV2-Env17 | Env 237-380 (gp120) |
| SabRV1-Env18 | SabRV2-Env18 | Env 335-498 (gp120) |
| SabRV1-Env20 | SabRV2-Env20 | Env 486-632 (gp120/gp41) |
| SabRV1-Env21L | SabRV2-Env21L | Env 526-698 (gp41, extra) |
| SabRV1-Env22 | | Env 712-879 (gp41, cyto) |
| SabRV1-Nef23 | SabRV2-Nef23 | Nef 1-145 |
| SabRV1-Nef24 | SabRV2-Nef24 | Nef 126-262 |
| | SabRV2-Tat25 | Tat 1-130 |

Viruses were mixed together such that, in the SabRV1-SIV cocktail, each virus (of the 20) was present at 2.5×10⁶ PFU/ml, giving a fmal concentration of 5×10⁷ PFU/ml. The SabRV2-SIV cocktail was mixed such that each virus (of the 20) was present at 5×10⁴ Pfu/ml, giving a final concentration of 1×10⁶ PFU/ml. The cocktails were made using pure P₀ viral stocks.

SlV_{mac}251 stock used for challenge was from May 1998, and has not been previously published. The SIV_{mac}251 (May 1998) stock has a titer of>10⁵ TCID₅₀ per 1 ml.

### Reverse Transcription-Polymerase Chain Reaction (RT-PCR) of Recombinant Polioviruses

2-5×10⁵ HeLa cells in 6-well dishes were infected with an MOI of 0.5-10 of the appropriate virus (an MOI of 10 was used if available). Cells were incubated at 37° C. in 1 ml of DMEM/F12+10% FCS for 6-8 hrs, and then harvested by scraping or trypinization. RNA was collected using RNeasy (Qiagen) and cDNA was synthesized using random primed Superscript II (Life Technologies) reactions. PCR was done using rTth (Perkin Elmer XL polymerase) and primers S1-3240F and S1-3580R (MoV2.11, S1, and SabRV1 recombinants) or primers S2-3151F and S2-3518R (S2-10F and SabRV2 recombinants). Conditions were: 0.5 μt cDNA, 2.2 mM Mg(OAc)₂, 0.5 μl XL polymerase, and manufacturer recommended buffer and primer concentrations in a 50 μl reaction, with 94° C. for 1 min, 50° C. for 1 min, and 72° C. for 1 min with 30 cycles. Generally 1-5 μl of the final product was loaded on a 1.5% agarose gel for analysis.

### Animals

All animals used in this study were mature, cycling, female cynomolgus macaques from the California Regional Primate Research Center. The animals were housed in accordance with American Association for Accreditation of Laboratory Animal Care standards. When necessary, animals were immobilized with 10 mg of ketamine HCl (Parke-Davis, Morris Plains, N.J.) per kg of body weight injected intramuscularly. The investigators adhered to the Guide for the Care and Use of Laboratory Animals prepared by the Committee on Care and Use of Laboratory Animals of the Institute of Laboratory Resources, National Resource Council. Prior to use, animals were negative for antibodies to HIV-2, SIV, type D retrovirus, and simian T cell leukemia virus type 1.

Intranasal inoculations of SabRV1-SIV and SabRV2-SIV were done in a total volume of 1 ml. The animals were anesthetized and placed in dorsal recumbancy with the head tilted back. One half ml of virus was instilled dropwise into each nostril. The animals were kept in this position for 10 min and then placed in lateral recumbancy until recovery from the anesthesia. Imaoka et al. (1998) J. Immunol. 161:5952-5958. Seven animals were inoculated intranasally with 1 ml (5×10⁷ PFU) of SabRV1-SIV on days 1, 3, 14, and 16, for a total of four immunizations.

Nineteen weeks after the first series of inoculations, these same seven animals were boosted with two intranasal inoculations of 1 ml (1×10⁶ PFU/ml) SabRV2-SIV, one on week 19, and a second at week 21. Intranasal inoculations were done because cynomolgus macaques can be consistently infected with poliovirus by this route (Crotty et al. (1999) J. Virol. 73:9485-9495), and also, macaque experiments with the model antigen cholera toxin indicate that intranasal immunization is better at eliciting vaginal immune responses than oral immunization. lmaoka et al. (1998) J. Immunol. 161:5952-5958.

The animals were challenged with 10⁵ TCID₅₀ of SIV_{mac}251 intravaginally using the SIV_{mac}251 (May 1998) stock (see above). A total of 2 intravaginal SIV inoculations were given to each monkey in a single day, with a four hour rest period between the inoculation procedures. Procedure and technique used were the same as previously described. Miller et al. (1997) J. Virol. 71:1911-1921.

### Serum and Vaginal and Rectal Lavage Antibody Responses

Anti-SIV IgG and IgA responses in vaginal and rectal washes and serum were measured at weekly timepoints during the study. Vaginal and rectal wash samples were collected and analyzed as previously described. Lu et al. (1999) Infect. Immun. 67:6321-6328; Lu et al. (1997) AIDS 12:1-10; and Miller et al. (1997) J. Virol. 71:1911-1921. Briefly, vaginal washes were collected by infusing 2 ml of sterile PBS into the vaginal canal and aspirating the instilled volume. Rectal washes were collected in a comparable manner. Samples were immediately snap-frozen on dry ice and stored at −80° C. until analysis.

To account for the presence of IgG interfering with and reducing the detection of IgA, sera was first depleted of IgG using Protein G-sepharose beads (Pharmacia Biotech, Uppsala, Sweden) prior to use in the IgA ELISA. To deplete IgG, 25 μl of serum sample was incubated with 100 μl Protein G-sepharose beads for 1 hr at 37° C. and then 4° C. overnight, and then the Protein G-sepharose was pelleted and the supernatant was collected. Dilution of sample during this process was 1:3. The ΔOD between test and control wells was defined as the difference between the mean OD of sample tested in two antigen-coated wells and the mean OD of the sample tested in two antigen-free control wells. The negative control OD value was determined from 12 uninfected monkey serum samples and defined stringently as the average OD plus 3 standard deviations.

Endpoint titers were determined if the ΔOD of the test sample exceeded the negative control value by a factor of 2. To then quantify anti-SIV antibody titers, serial four-fold dilutions of duplicate samples of sera, vaginal wash, or rectal wash were tested by ELISA using whole pelleted SIV_{mac}251 (Advanced Biologics Inc., Columbia, Md.). Antibody binding was detected with peroxidase conjugated goat anti-monkey-IgG(Fc) or -IgG(Fc) (Nordic Laboratories, San Juan Capistrano, Calif.) and developed with o-phenlyenediamine dihydrochloride (Sigmna). The endpoint titer was defined as the reciprocal of the last dilution giving an ΔOD greater than 0.1. Crotty et al. (1999) J. Virol. 73:9485-9495.

### Neutralizing Antibody Responses

Assays were done as previously described (Benson et al. (1998) J. Virol. 72:4170-4182; and Montefiori et al. (1996)J. Immunol. 157:5528-5535); neutralizing antibody titers are the dilution at which cell killing by lab adapted SIV_{mac}251 was inhibited 50%.

### SIV Virus Isolation and Serum Viral RNA Load Determination

Virus was isolated from heparinized whole blood obtained from the SIV-oculated cynomolgus macaques. PBMC were isolated by Ficoll gradient separation ymphocyte Separation Medium, Organon Teknika, West Chester, Pa.) and co-cultured with CEMx174 cells (Hoxie et al. (1988) J. Virol. 62:2557-2568), (provided by James A. Hoxie, University of Pennsylvania, Philadelphia) as previously described. Lohman et al. 1991) J. Clin. Microbiol. 29:2187-2192. Five million PBMC were co-cultivated with 2-3 million CEMx174 cells. Aliquots of the culture media were assayed regularly for the esence of SIV major core protein (p27) by antigen capture ELISA. Lohman et al. (1991) J. Clin. Microbiol. 29:2187-2192. Cultures were considered positive if they were antigen positive at 2 consecutive time points. A detailed description of the technique and criteria to determine if culture media was antigen positive has been published. Marthas et al. (1993) J. Virol. 67:6047-6055. All cultures were maintained for 8 weeks and tested for SIV p27 by ELISA before being scored as virus negative. Blood samples for virus isolation were collected at the times indicated in Table 2.

TABLE 2
SIV virus isolation

| week^{a} | 1 | 2 | 4 | 6 | 8 | 12 | 16 |
|---|---|---|---|---|---|---|---|
| 27244 | − | − | − | − | − | − | − |
| 27270 | − | − | + | − | − | − | − |
| 25231 | + | + | + | + | + | + | + |
| 27250 | + | + | + | + | + | + | + |
| 28508 | + | + | + | + | + | + | − |
| 27253 | + | + | + | + | + | + | + |
| 27273 | + | + | + | + | + | + | + |
| 26385 | + | + | + | + | + | + | + |
| 28118 | + | + | + | + | + | + | + |
| 26560 | + | + | + | + | + | − | + |
| 26383 | − | + | + | + | + | + | + |
| 26405 | − | + | + | + | + | + | + |
| 23414 | − | + | + | + | + | + | + |

^{a}Upper seven monkeys are vaccinated, lower six are control monkeys

SIV RNA loads were determined using a modification (Lifson et al., in reparation) of a real time RT-PCR assay on monkey plasma samples, essentially as reviously described. Suryanarayana et al. (1998) AIDS Res. Hum. Retrovir. 14:183-189. The assay has a threshold sensitivity of 100 copy Eq/mL of plasma, and an interassay coefficient of variation of <25%.

### SIV Provirus PCR Analysis

Nested PCR was carried out on genomic PBMC DNA in a DNA Thermal Cycler (Perkin-Elmer Cetus, Emeryville, Calif.) as previously described. Miller et al. (1997) J. Virol. 71:1911 -1921. Briefly, cryopreserved PBMCs isolated from whole blood of each monkey in the experiment were washed 3 times in Tris buffer at 4° C. and resuspended at 10⁷ cells/ml. Ten microliters of the cell suspension were added to 10 microliters of PCR lysis buffer (50 mM Tris-HCl (pH 8.3), 0.45% NP-40, 0.45% Tween-20) with 200 ug/ml Proteinase K. The cells were incubated for 3 hours at 55° C., followed by 10 minutes at 96° C. Two rounds of 30 cycles of amplification were performed on aliquots of plasmid DNA containing the complete genome of SIV_{mac}1A11 (positive control) or aliquots of cell lysates using conditions described elsewhere. Miller et al. (1997) J. Virol. 71:1911 -1921. The primers used specifically amplify SIV Gag. DNA from uninfected CEMx174 cells was amplified as a negative control in all assays to monitor potential reagent contamination. β-actin DNA sequences were amplified with 2 rounds of PCR (30 cycles/round) from all PBMC lysates to detect potential inhibitors of Taq polymerase. Following the second round of amplification, a 10 μl aliquot of the reaction product was removed and run on a 1.5% agarose gel. Amplified products in the gel were visualized by ethidium bromide staining. Blood samples for PCR analysis were collected at the times indicated in Table 3.

TABLE 3
SIV proviral DNA PCR

| week^{a} | 1 | 2 | 4 | 8 | 12 |
|---|---|---|---|---|---|
| 27244 | − | − | − | − | − |
| 27270 | − | − | − | − | − |
| 25231 | − | + | − | + | + |
| 27250 | − | − | + | + | + |
| 28508 | − | − | + | + | + |
| 27253 | − | − | + | + | + |
| 27273 | − | − | + | + | + |
| 26385 | − | − | − | + | + |
| 28118 | − | − | − | + | + |
| 26560 | − | + | + | + | + |
| 26383 | − | + | − | + | + |
| 26405 | − | − | − | + | + |
| 23414 | − | nd | + | + | + |

^{a}Upper seven monkeys are vaccinated, lower six are control monkeys
nd = not done
Nested PCR used gag specific primers.

### Western Blot Analysis of Serum Antibody Responses

2×10⁶ HeLa cells infected with wild type poliovirus were incubated for 7 hours at 37 C. Cells were harvested and lysed on ice for 1 min (lysis buffer consisted of 10 mM Tris (pH 7.5), 140 mM NaCl, 5 mM KCl, and 1% IGEPAL), and nuclei were removed by centrifugation. 20 μl of polio-infected whole-cell lysate and 22 μg of sucrose-gradient purified SIV_{mac}251 (ABI, Columbia, Md.) were electrophoresed in parallel lanes through a 10% or 12% SDS-polyacrylamide gel and analyzed by immunoblotting. The anti-SIV serum used as a positive control for SIV proteins was pooled serum from SIV-infected rhesus macaques.

The anti-polio serum used as a positive control for poliovirus proteins was obtained from a polio-immune human. Antisera from all vaccinated cynomolgus macaques were used as primary antibody (serum from day of challenge was used for monkeys 27270, 27244, 28508, 27273, 27253 and serum from one month pre-challenge was used for monkeys 25231 and 27250). Also, pre-immune serum from monkey 27250 was used. Secondary antibody (horseradish peroxidase-conjugated rabbit anti-human IgG) was obtained from DAKO (Carpinteria, Calif.) and used for monkeys 27270, 27244, 28508, 27273, 27253, and 27250-pre-immune).

A horseradish peroxidase-conjugated rabbit anti-rhesus monkey IgG (Sigma, Saint Louis, Mo.) was used for monkeys 25231 and 27250, using one month pre-challenge serum. Blots were probed with 1:100-diluted monkey serum in TBST (10 mM Tris-HCl (pH 7.5), 150 mM NaCl, 0.15% Tween-20) with 10 % fat-free dry milk (Biorad, Hercules, Calif.), washed twice in TBST containing 0.15% Tween-20 and once in TBST containing 0.5% Tween 20, probed with the secondary antibody (1:2,000 dilution), and then detected by enhanced chemiluminescence (ECL; Amersham, Arlington Heights, Ill.) as specified by the manufacturer. Rhesus monkey serum was used at a dilution of 1:200 and polio-immune human serum was used at a dilution of 1:25. Films were digitally scanned and exported to Photoshop 5.5 (Adobe, San Jose, Calif.).

### Statistical Methods

SIV viremia levels were analyzed by Student's T-test comparison of the 24-32 week average log viral load of each animal in the two groups (vaccinated:control) with two-tailed distribution. Weight gain (or loss) was analyzed by Mann-Whitney rank test of the 33-44 week average weight change (from day of challenge) of each animal in the two groups (vaccinated:control) with two-tailed distribution. Mortality differences between the two groups at week 48 were analyzed by Fisher's exact test.

### Lymphocyte Proliferative Responses to SIV Antigens

Antigen specific proliferation was tested using PBMC from fresh blood samples as described. McChesney et al. (1998) J. Virol. 72:10029-10035. The cells were suspended at 2×10⁶ per ml in RPMI 1640 medium supplemented with 10% FCS and plated in triplicate at 50 μl per well in 96-well round bottom microtiter plates. Antigen dilutions or control reagents were plated at 50 μl per well. 100 μl fresh medium was added after 48 hrs and the plates were incubated for 7 days in a CO₂ incubator. The wells were pulsed with ³H-thymidine (1 μCi per well, NEN-DuPont Co., Wilmington, Del.) overnight prior to harvest. The plates were aspirated onto fiberglass filters and washed with a cell harvester (Inotech Biosystems International, Lansing, Mich.).

The filters were saturated with scintillation cocktail and sealed, and counts in the ³H window were measured using a 96-well scintillation counter (Microbeta 1450, Wallac Biosystems, Gaithersburg, Md.). The SIV antigen was whole-inactivated SlV_{mac}239 (kindly provided by Dr. Larry Arthur). ConA was tested as a positive control antigen. Medium alone was the control for spontaneous proliferation. The antigens were tested at 0.1, 1.0 and 10 μg/ml in every assay. This assay was used in our previous study. Crotty et al. (1999) J. Virol. 73:9485-9495. Lymphocyte proliferation assays were performed before immunization and at regular intervals after immunization using PBMCs. Due to an unusually high level of spontaneous proliferation (˜10,000-100,000 cpm/well) in negative control wells (PBMCs plus medium alone) at all time points tested over a 28 week period, it was difficult to assess SIV-specific CD4⁺ T cell responses in the immunized animals, as minimal additional stimulation was seen in the presence of antigen or Con A.

### SIV-Specific CTL Responses

The presence of SIV specific CTLs in cynomolgus PBMCs was assessed as previously reported. Crotty et al. (1999) J. Virol. 73:9485-9495. Briefly, PBMCs from immunized monkeys were stimulated with 10 μg/ml Con A (Sigma) and cultured for 14 days in complete medium supplemented with 5% human lymphocyte-conditioned medium (Hu IL-2, Hemagen Diagnostics, Waltham, Mass.). Autologous B cells were transformed by Herpes papio (595S×1055 producer cell line, provided by M. Sharp, Southwest Foundation for Biomedical Research, San Antonio, Tex.), and infected overnight at an MOI of 30 with wild-type vaccinia virus (vvWR), or recombinant vaccinia expressing the p55^{gag} (vv-gag) or gp160^{env} (vv-env) of SlVmac239 (provided by L. Giavedoni and T. Yilma, University of California, Davis, Calif.).

The level of vaccinia virus infection of target cells was estimated by indirect immunofluorescence using monkey anti-vaccinia virus antibody, followed by fluoresceinated goat anti-human IgG (Vector Laboratories, Burlingame, Calif.). The level of vaccinia virus infection of target cells in this series of experiments was estimated to fall between 5 and 15%. Target cells were labeled with 50 μCi of⁵¹Cr (Na₂CrO₄, Amersham Holdings, Arlington Heights, Ill.) per 10⁶ cells. Effector and target cells were added together at multiple E:T ratios in a 4 hr chromium release assay. Assays were considered reliable if specific lysis was >10% and at least twice the level of spontaneous lysis of vvWR infected cells. At many time points the data from the CTL assays could not be meaningfully interpreted due to high spontaneous lysis of the cynomolgus macaque transformed-B cell targets. Lysis was not due to NK cell activity, as no lysis was seen when the NK target cell line K562 was substituted as a negative control. Numerous variations of the CTL assay were attempted to generate consistently reliable chromium release assay data in immunized or infected cynomolgus macaques. Cold-target inhibition, a variety of stimulation procedures, and enrichment of CD8⁺ cells using anti-CD8 bead purification failed to consistently resolve this problem.

### RESULTS

### Sabin-Based Vaccine Vector Construction and Production

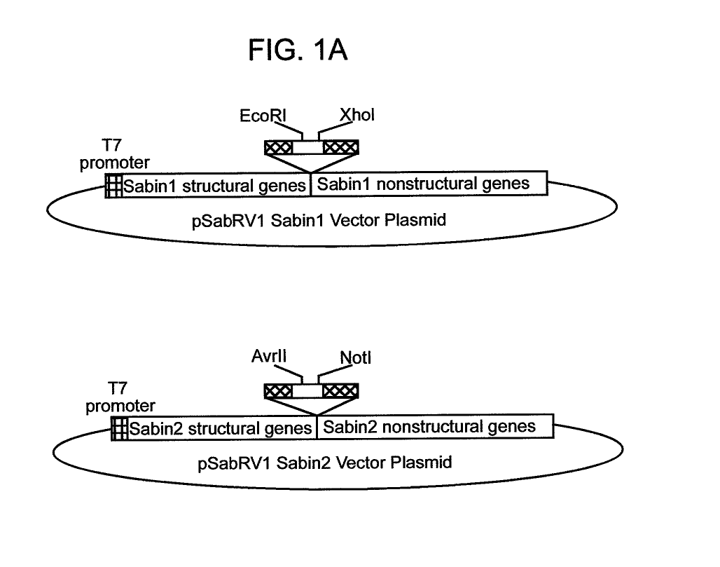

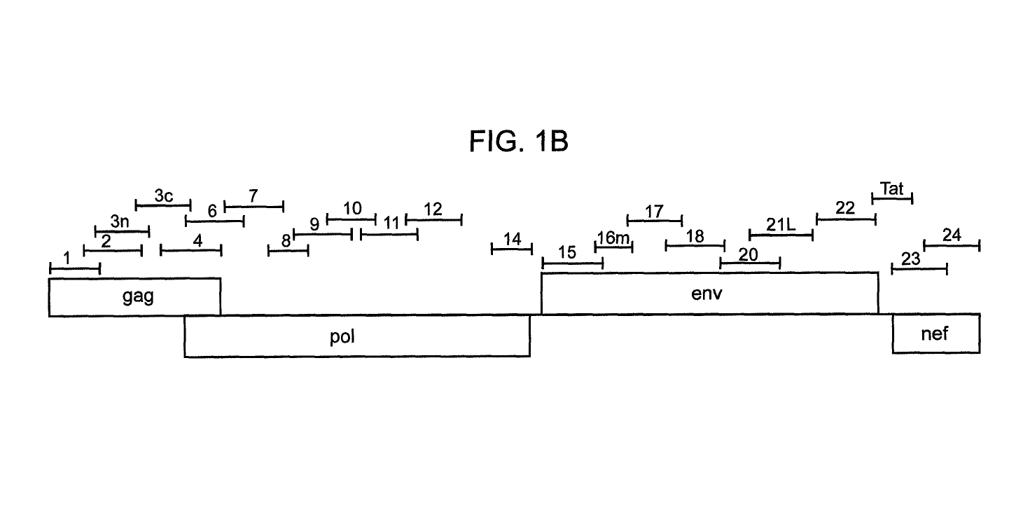

Given the excellent safety record of Sabin vaccine strain polioviruses in humans (Sutter et al. (1999) In S. Plotkin an W Orenstein, eds. Vaccines 3^{rd} edition, W. B. Saunders, Philadelphia, page 1230 “Live attenuated poliovirus vaccines”), we used only Sabin based viruses produced from molecularly defined constructs. Hence, we engineered new plasmid clones of Sabin 1 and Sabin 2 derived vectors (pSabRV1 and pSabRV2), (FIG. 1A). We then constructed a collection of 20 SabRV1 viruses expressing SIV gag, pol, env, and nef that represent nearly the entire SIV genome (FIG. 1B and Table 1). These viruses grew well, as assessed by plaque assay. Twenty SabRV2-SIV viruses were produced in a comparable manner, selected to represent a similar coverage of the major SIV genes, plus Tat (FIG. 1B and Table 1).

These viruses also grew well, as assessed by plaque assay. In some cases, one difficulty with the use of recombinant polioviruses is producing genetically pure stocks, since viruses with deletions in their insert sequences can accumulate as recombinant polioviruses replicate through a number of generations. Crotty et al. (1999) J. Virol. 73:9485-9495; Mueller et al. (1998) J. Virol. 72:20-31; and Tang et al. (1997) J. Virol. 71:7841-7850. Therefore we took great care to check the viral stocks for deletions. We did so by using a sensitive RT-PCR assay capable of detecting deleted virus comprising as little as 0.1% of the stock. The SabRV1-SIV vaccine stocks were greater that 99.9% pure in total, as were the SabRV2-SIV stocks. To further test the viability and stability of the recombinant viruses, a cocktail of nine of the SabRV2-SIV viruses was then passaged repeatedly and assessed by RT-PCR. The vaccine cocktail as a whole maintained the SIV sequences for at least ten generations, and all of the individual component viruses maintained their inserts and viability.

The twenty SabRV1-SIV virus stocks were then mixed to create a defined vaccine cocktail of 5×10⁷ PFU/ml for use in the primate vaccinations described below.

The twenty SabRV2-SIV virus stocks were mixed to make a defined vaccine cocktail of 1×10⁶ PFU/ml.

### Monkey Immunizations

Producing a candidate SIV vaccine in two different serotypes of poliovirus (type 1 and type 2 strains) was a strategy employed in our last macaque study to create a better opportunity for an effective booster immunization using the second vector serotype, as there is no significant cross-neutralization or cross-protection between these two serotypes. That approach resulted in anemnestic booster responses in the immunized animals (Crotty et al. (1999) J. Virol. 73:9485-9495), and led us to utilize the same strategy in the study reported here.

Cynomolgus macaques are used in our studies because they are orally and intranasally susceptible to poliovirus. As the goal of our studies is to test poliovirus vectors as potential mucosal AIDS vaccines, we used a route of inoculation that would elicit a vaginal immune response. An intranasal route of inoculation was chosen for these experiments because previous experiments using SIV subunits plus cholera toxin have demonstrated that the intranasal route of inoculation elicits a better vaginal mucosal immune response than oral or rectal immunization. lmaoka et al. (1998) J. Immunol. 161:5962-5958.

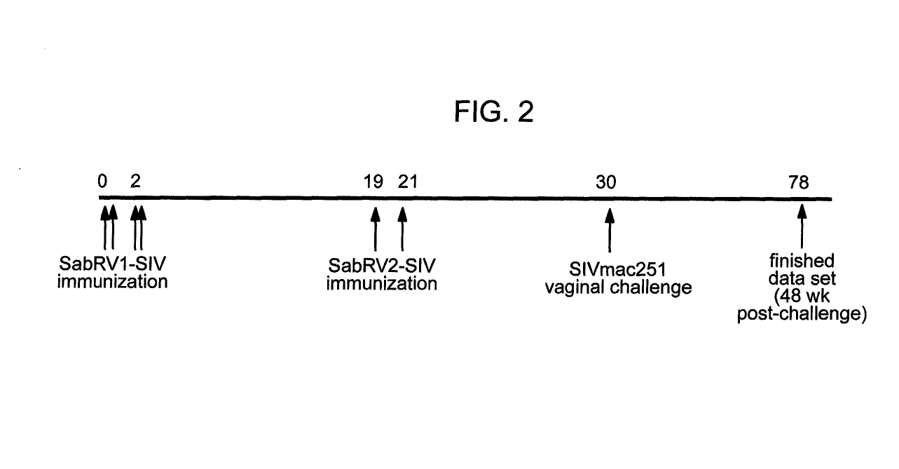

In this study, seven cynomolgus macaques were immunized intranasally with SabRV1-SIV (5×10⁷ PFU) at week 0 and 2 (FIG. 2). Then at week 19 and 21, these animals were booster immunized intranasally with SabRV2-SIV (1×10⁶ PFU). These doses are comparable to normal Sabin oral poliovirus vaccine (OPV) doses used in children. AFHS (1998) AFHS Drug Information. American Society of Hospital Pharmacists: Silver Platter International, Bethesda, Md.

### Immunization Induces Strong Serum Anti-SIV IgG and IgA Responses

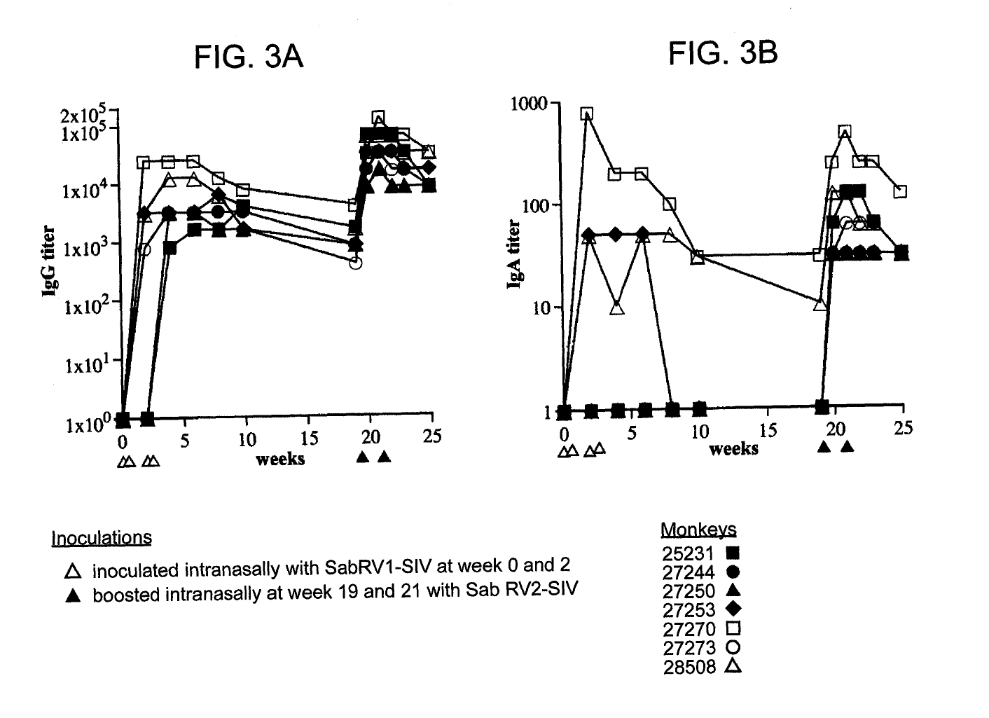

The results of ELISAs for serum IgG and IgA responses against SIV are shown in FIG. 3A and FIG. 3B. All seven monkeys made a rapid and strong anti-SIV IgG response after immunization with SabRV1-SIV (FIG. 3A). Three of the seven monkeys made serum anti-SIV IgA responses and in two of those animals the SabRV1-SIV elicited IgA responses persisted for at least 19 weeks (FIG. 3B).

The SabRV2-SIV booster immunization at 19 weeks resulted in a 20-80× increase in anti-SIV IgG antibody titers in all monkeys within 7 days, a classic anamnestic antibody response (FIG. 3A). Additionally, all 7 monkeys were positive for anti-SIV serum IgA at one week post-boost, with a greater than 10× titer increase in all monkeys, confirming the presence of an anamnestic IgA response in all vaccinated monkeys (FIG. 3B).

All seven monkeys made comparable serum IgG anti-SIV antibody titers; and, generally, the monkeys made comparable serum IgA anti-SIV antibody titers after the SabRV2-SIV booster immunization. Individual variability in the immune response to the vaccine was seen; monkey 27270 made the strongest anti-SIV serum IgG and IgA response after both the SabRV1-SIV and SabRV2-SIV immunizations (FIG. 3A and FIG. 3B).

### Immunization Induces Vaginal and Rectal Anti-SIV Antibody Responses

In this study, we analyzed antibody samples taken from the vaginal and rectal mucosal surfaces. It was recently shown that macaque vaginal antibody secretions are affected by the menstrual cycle (Lu et al. (1999) Infect. Immun. 67:6321-6328), and therefore in this study we took mucosal antibody samples on a weekly basis, to assess the antibody levels more accurately.

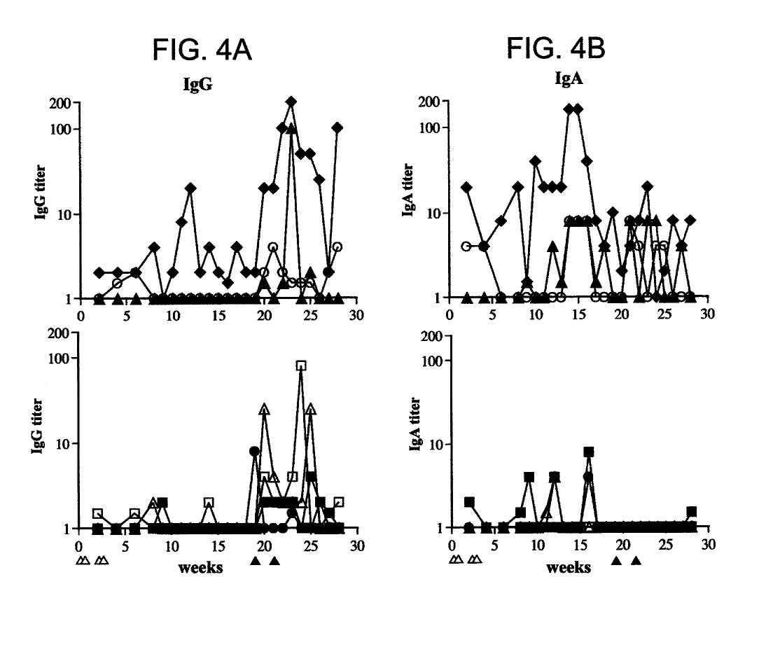

In this study, we observed that 100% of the immunized monkeys made rectal mucosal anti-SIV antibody responses. After the SabRV1-SIV immunization, all seven monkeys made at least transient anti-SIV rectal IgA responses, even though only three had made detectable serum anti-SIV IgA (FIG. 3B; FIG. 4B). Conversely, neither of the two monkeys that made long-lasting serum IgA responses (27270 and 28508, FIG. 3B) after SabRV1-SIV immunization in the current study, made detectable rectal IgA antibodies for longer than 2 weeks.

We detected anti-SIV IgG in rectal secretions from all seven monkeys after the w SabRV2-SIV immunization (FIG. 4A). It is uncommon to observe IgG in rectal secretions. Lu et al. (1999) Infect. Immun. 67:6321-6328; Ogra et al. (1999) Mucosal Inmunity 2^{nd} ed. Academic Press, San Diego. Monkeys 27253 and 27270 made particularly strong, 50-100× anamnestic rectal IgG responses after the SabRV2-SIV immunization. Additionally, we were intrigued that the SabRV2-SIV immunization appeared to reduce rather than boost the rectal IgA anti-SIV response in the monkeys (FIG. 4B). Four monkeys that made a transient rectal IgA response after the SabRV1-SIV immunization (25231, 28508, 27244, and 27270), had no detectable anti-SIV rectal IgA after the SabRV2-SIV immunization (FIG. 4B), even though all four monkeys had a substantial increase in serum anti-SIV IgA titers (FIG. 3B).

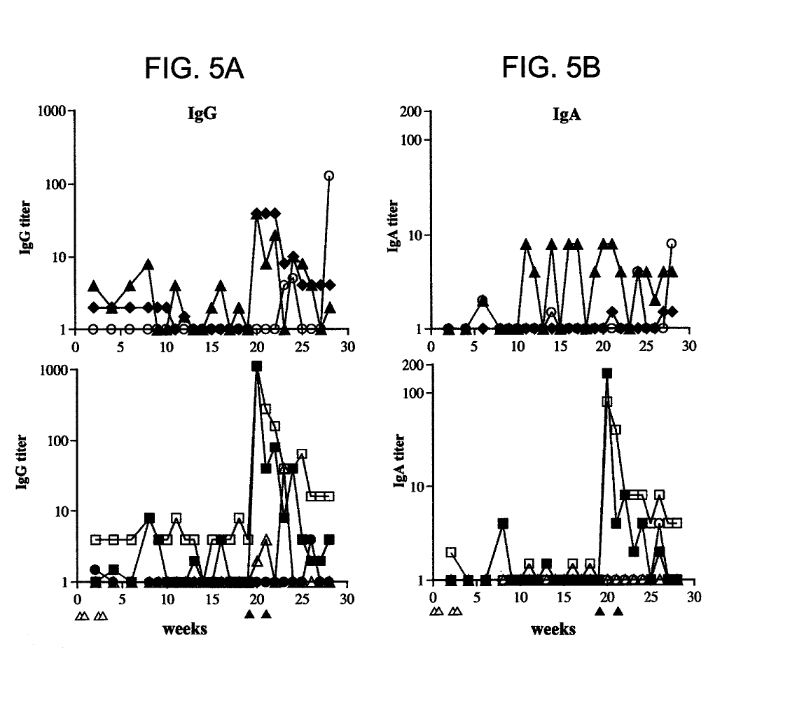

All of the monkeys made vaginal IgG anti-SIV responses after SabRV1/2-SIV immunization, and six out of seven monkeys made vaginal IgA anti-SIV responses (FIG. 5A and FIG. 5B). Interestingly, the monkey with the most substantial vaginal IgA antibody response (27250) after SabRV1-SIV immunization (FIG. 5B) did not make a concurrent serum IgA response (FIG. 3B). This animal also had a robust rectal IgA response (FIG. 4B), providing more evidence for compartmentalization of the immune responses to the vaccine in some animals.

As with rectal antibodies, the strongest vaginal IgG antibody responses occurred after the SabRV2-SIV booster immunization. A 200-1000× increase in vaginal anti-SIV IgG was seen in monkeys 27270 and 25231 after the booster immunization. A 100× increase in vaginal IgA anti-SIV antibodies was seen in these same two monkeys. For each individual monkey, the pattern of vaginal IgA and IgG anti-SIV responses were general similar in profile (FIG. 5A and FIG. 5B).

Taken together, though all monkeys made similar serum IgG anti-SIV antibody responses, and similar serum IgA responses after the booster immunization, there were substantial differences in the mucosal antibody responses of different monkeys. In animals with a strong initial rectal IgA response (monkeys 27253 and 27273, FIG. 4B), vaginal IgA responses were weak or absent. SabRV2 appeared to elicit rectal IgG antibody responses but not rectal IgA responses, as noted above. The three monkeys that made anti-SIV serum IgA responses after SabRV1-SIV immunization made the strongest rectal IgG responses after the SabRV2-SIV booster immunization, but the significance of this is unclear. The results of these experiments clearly demonstrate that serum antibody titers are not a good indicator of mucosal antibody responses, consistent with compartmentalization of the immune response.

### Diversity of Antigens Recognized in SabRV1/2-SIV Immunized Monkeys

We are unaware of any precedent for immunization of primates with a viral vector (or any vector) expressing a defined library of antigens. Therefore, it was important to determine whether the measured antibody responses were against a single antigen (expressed by a single SabRV-SIV virus) or multiple antigens (expressed by different SabRV-SIV viruses). To explore this issue, we examined the anti-SIV and anti-polio specificities of the serum antibodies in immunized animals by western blotting. All seven monkeys seroconverted to poliovirus antigens, generally with a strong response against capsid protein VP1 and weaker responses against two to four other poliovirus proteins. All seven monkeys also seroconverted to SIV antigens by western blot, confirming the SIV ELISA results in FIG. 3A and FIG. 3B. Importantly, a majority of monkeys made substantial antibody responses to multiple SIV proteins.

Antibody responses against RT (p51/65) (all seven monkeys), Gag (p55 (six monkeys: 27244, 28508, 27270, 27273, 27253, and 27250); p17 (monkeys 27270, 27253); and p27 (monkeys 27244, 27250)), Env gp41 (monkeys 27270, 27253), and Env gp120 (monkeys 27244, 28508, 27270, 25231, 27253) were all apparent. Antibodies against Nef and Tat (also represented in the SabRV1-SIV and/or SabRV2-SIV cocktail) are not assayed in this experiment, as they are not packaged in SIV virions, which is the target material for the immunoblots. At least five different SabRV1/2-SIV viruses, and possibly many more, were immunogenic and elicited antibody responses, as the responses against SIV p27, RT, p17, gp41, and gp120 must have been elicited from different SabRV-SIV's. In summary, a majority of monkeys responded to multiple poliovirus and SIV proteins, indicating that the library vaccine approach is successful at eliciting responses to multiple expressed proteins, even in a complex cocktail of twenty different viruses.

### Cellular Immune Responses

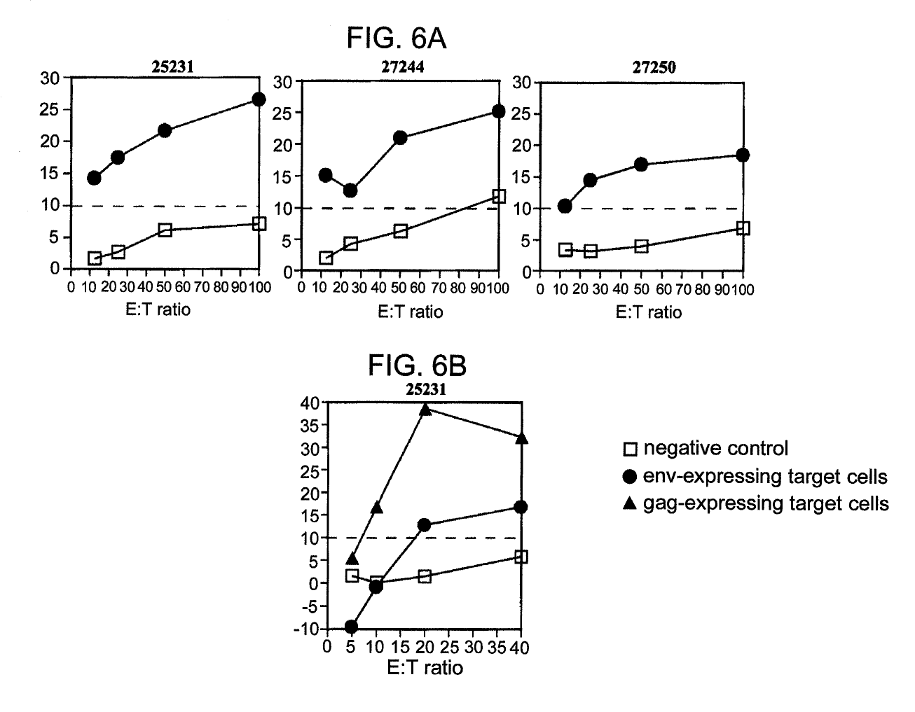

Poliovirus vectors can elicit potent cytotoxic T lymphocyte (CTL) responses in both mice and primates. Mandl et al. (1998) Proc. Natl. Acad. Sci. USA 95:8216-8221; Singh et al. (1999) J. Virol. 73:4823-4828; and Crotty et al. (1999) J. Virol. 73:9485-9495. Here we were able to detect SIV Env specific CTLs in 3 of 7 monkeys after SabRV1-SIV vaccination (25231, 27244, 27250) by a standard bulk PBMC cytolytic assay (FIG. 6A). After the SabRV2-SIV vaccinations we detected SIV Gag and Env specific CTLs in monkey 25231 (FIG. 6B). Cellular immune responses are technically difficult to assess in cynomolgus macaques (see Materials and Methods), and we frequently experienced difficulties with high background lysis. This technical complication prevented accurate assessment of CTL activity at additional time points.

The three monkeys that tested positive for SIV-specific CTLs after SabRV-SIV vaccination (25231, 27244, 27250) also tested positive for SIV-specific lymphoproliferative responses (S.I. of 3.3, 4.1, and 2.7 respectively).

### Virologic Outcome of Vaginal Challenge With SIVmac251

All seven vaccinated monkeys and a total of 12 control monkeys were challenged with a vaginal inoculum of SIV_{mac}251. SIV_{mac}251 is an uncloned and highly virulent virus that has proven to be extremely difficult to protect against (i.e. prevent infection) or control with vaccine induced immune responses. Buge et al. (1997) J. Virol. 71:8531-8541; Daniel et al. (1994) AIDS Res. Hum. Retrovir. 10:839-851; Giavedoni et al (1993) J. Virol. 67:577-583; Hanke et al. (1999)J. Virol. 73:7524-7532; Lu et al. (1996) J. Virol. 70:3978-3991; Lu et al. (1997) AIDS 12:1-10; and Schlienger et al. (1994) J. Virol. 68:6578-65-88. The vaginal challenge route was chosen because our primary interest in the SabRV vector is as a vaccine capable of protecting against sexually transmitted HIV.

Six control cynomolgus macaques were first challenged intravaginally with 1×10⁵ TCID₅₀ SlV_{mac}251 twice in one day (this dose had previously infected 15 of 15 rhesus macaques intravaginally). All 6 of those control cynomolgus macaques became infected, as judged by positive SIV virus isolation, positive SIV provirus PCR, and seroconversion to SIV antigens.

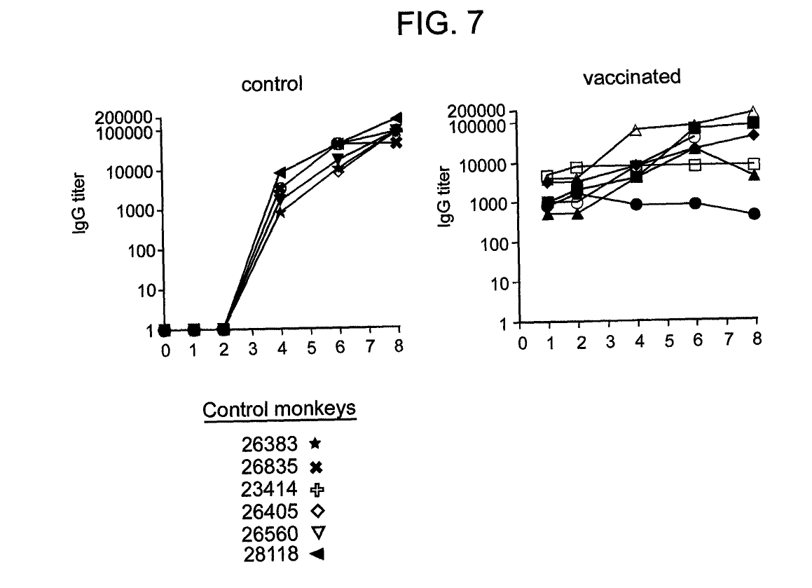

At week 30, nine weeks after the last immunization, we challenged all seven of the SabRV-SIV vaccinated animals, and six additional concurrent control cynomolgus macaques, with two vaginal inoculations of 1×10⁵ TCID₅₀ SlV_{mac}251 in one day (FIG. 2). All six concurrent control monkeys became SIV⁺ by virus isolation (Table 2), SIV provirus PCR (Table 3), and seroconversion to SIV (neutralizing antibodies, Table 4; ELISA, FIG. 7), bringing the total to 12 of 12 control cynomolgus macaques infected after vaginal inoculation with the challenge dose.

TABLE 4
Neutralizing Antibodies

| Monkey^{a} | 0^{b} | 30 (Ch + 0)^{c} | 32 (Ch + 2) | 38 (Ch + 8) |
|---|---|---|---|---|
| 27244 | —^{d} | — | — | — |
| 27270 | — | — | — | — |
| 25231 | — | — | — | 7673 |
| 27250 | — | — | — | 1130 |
| 28508 | — | — | — | 6386 |
| 27253 | — | — | — | 421 |
| 27273 | — | — | — | 8348 |
| 26385 | — | — | — | 613 |
| 28118 | — | — | — | 1215 |
| 26560 | — | — | — | 1994 |
| 26383 | — | — | — | 512 |
| 26405 | — | — | — | 1764 |
| 23414 | — | — | — | 1301 |

^{a}Upper seven monkeys are vaccinated, lower six are control monkeys
^{b}Day of initial SabRV1-SIV immunization
^{c}Day of challenge. (Ch = challenge)
^{d}(—) indicates undetectable levels of neutralizing antibodies. See Materials and Methods. Titers indicated are reciprocal dilutions.

In the group of SabRV-SIV immunized monkeys, two of the monkeys appeared to be fully protected. SIV was never isolated from the PBMC of one animal (27244), while the other animal (27270) was SIV virus isolation positive at a single time point, 4 weeks post-challenge (Table 2). We were unable to detect SIV gag in PBMC samples from either animal by PCR (Table 3). Neither animal made an anamnestic serum antibody response to SIV antigens after the challenge exposure (FIG. 7), again indicating that they were fully protected from SIV infection.

All seven vaccinated monkeys and the six new control monkeys were assayed for SIV neutralizing antibody titers. No serum neutralizing antibody titers were detected before the vaginal SIV challenge in any animal (Table 4). No neutralizing antibody titers were seen post-challenge in vaccinated monkeys 27270 and 27244, again consistent with the SIV viral load data indicating that these two monkeys were fully protected from infection. Among the remaining monkeys, 4-fold higher neutralizing antibody titers were seen in the vaccinated monkeys versus the control monkeys post-challenge.

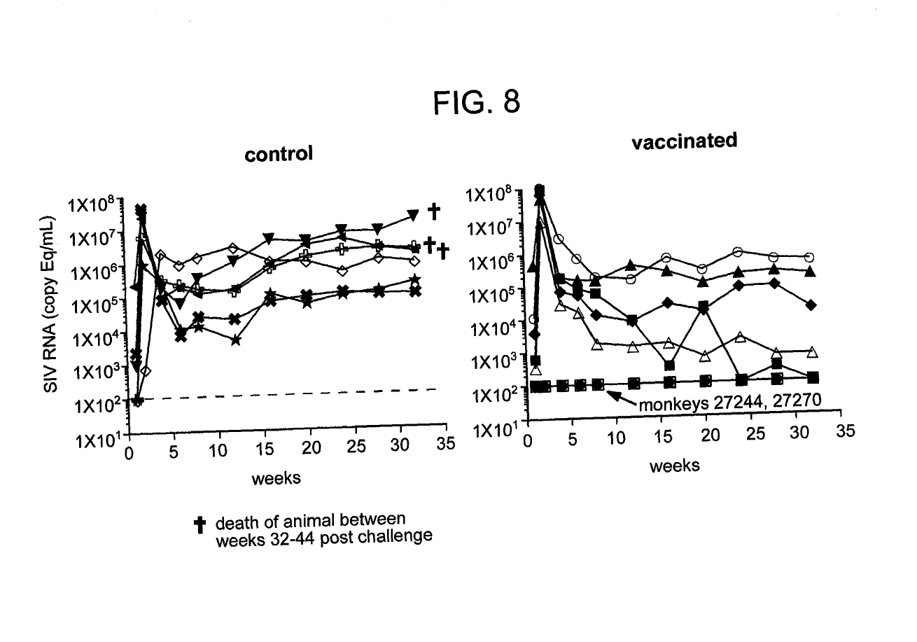

In order to quantify the SIV viremia in the challenged animals, a sensitive quantitative RT-PCR assay was used, which has been used in several macaque SIV studies (36, 53, 67). All six concurrent control monkeys had significant SIV viral loads, peaking between week 2-4 and reaching post-acute geometric mean viral loads of 9.3×10⁵ copies/ml by week 24-32 (FIG. 8).

SabRV-SIV vaccinated animals 27244 and 27270 had no detectable SIV RNA in plasma at any time point and confirming that these two animals were solidly protected (FIG. 10). Compared with the control monkeys, the seven SabRV1/2-SIV vaccinated monkeys had a 3.0 log₁₀ reduction in post-acute geometric mean viral load (P<0.01). Control of post-acute viremia was particularly obvious in two vaccinated monkeys: vaccinated monkey 28508 exhibited stable long term control of viremia to ˜1×10³ copies/ml, and vaccinated monkey 25231 reduced its SIV_{mac}251 viremia by more than 10⁶ fold during the post-acute phase, implicating a strong vaccine-elicited cellular immune response (FIG. 8).

### Clinical Outcome of Vaginal Challenge With SIV_{mac}251

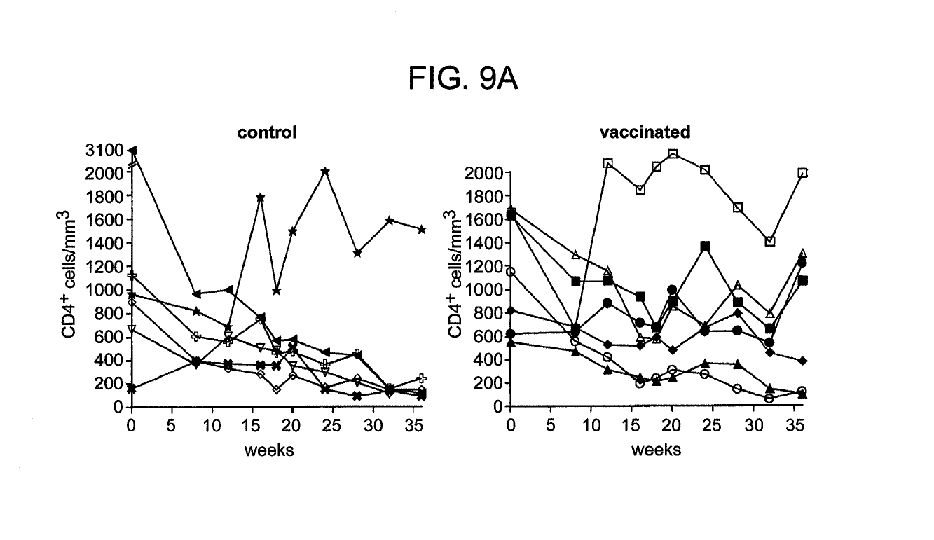

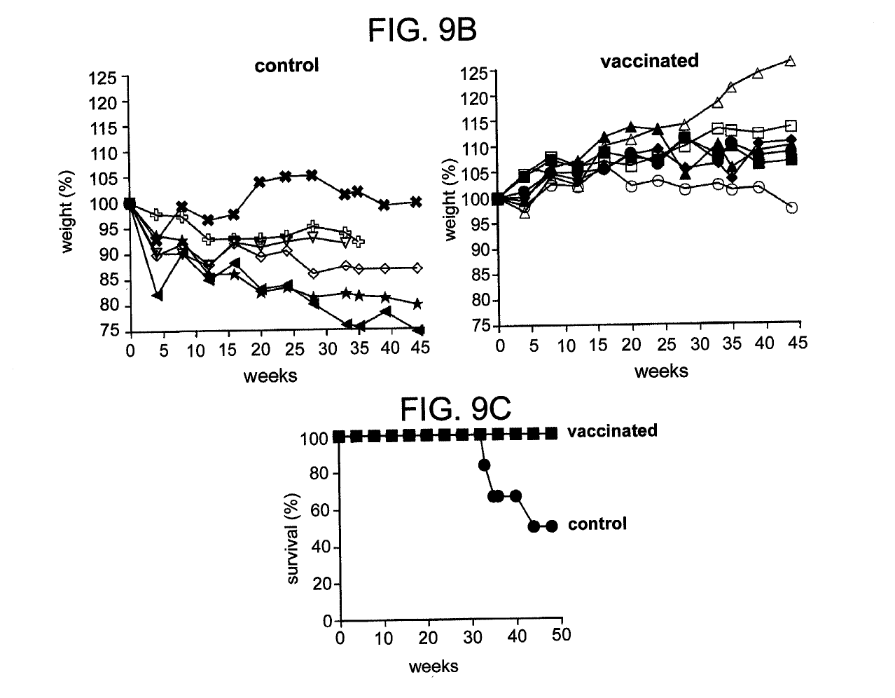

The clinical outcome of SIV infection was much worse in the control animals compared to the SabRV-SIV immunized animals. Five of 6 control animals had marked decreases in CD4⁺ T lymphocyte counts (FIG. 9A) and body weight (FIG. 9B) over the 48 week post-challenge observation period. Three of the 6 control animals were euthanized at 34, 35, and 44 weeks post challenge due to severe clinical AIDS (FIG. 9C). At necropsy, 2 of the animals (23414, 26560) had lymphoma and the other animal (28118) has severe non-responsive enteritis and wasting.

In sharp contrast, all 7 of the vaccinated monkeys are alive (P<0.07) and healthy (significantly better body weight, P<0.003, FIG. 9B) at 48 weeks post-challenge. Although CD4⁺ T cell counts declined initially after challenge, the counts stabilized at about 16 weeks post-challenge for 5 of the 7 vaccinated animals (FIG. 9A). Over the course of the study, the SabRV-SIV vaccinated animals had higher average CD4⁺ counts than the control animals. At 36 weeks post-challenge, the average CD4⁺ cell count of vaccinated animals was 840 cells/μl, while 5 of 6 control monkeys had CD4⁺ counts below 150 cells/μl. Two vaccinated animals (27250,27273) had depressed CD4⁺ T cell counts after challenge, consistent with their higher SIV viremia levels, but their body weights have remained stable (FIG. 9B) and they appear clinically normal. The other 5 animals have gained weight steadily since the vaccine challenge (FIG. 9B). These results demonstrate that the SabRV-SIV vaccine protects monkeys from SIV-related disease progression.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A population of live attenuated recombinant replication-competent polioviruses, wherein said population comprises at least two member viruses and wherein each of said member viruses comprises a nucleotide sequence encoding a different antigenic polypeptide from an organism other than poliovirus, which nucleotide sequence is capable of being expressed in a eukaryotic cell.

2. The population of claim 1, wherein said antigenic polypeptides are included within polyprotein precursors which are proteolytically processed to release said antigenic polypeptides.

3. The population of claim 1, wherein said antigenic polypeptides have a length of from about four to about 400 amino acids.

4. The population of claim 1, wherein the antigenic polypeptides are human immunodeficiency virus (HIV) polypeptides.

5. The population of claim 4, wherein said population expresses at least three different antigenic polypeptides.

6. The population of claim 4, wherein said population expresses at least four different antigenic polypeptides.

7. The population of claim 4, wherein said HIV is HIV type-1.

8. The population of claim 4, wherein said at least two different antigenic polypeptides are selected from the group consisting of gag, env, pol and nef polypeptides from HIV.

9. The population of claim 6, wherein said population comprises nucleotide sequences encoding overlapping fragments of the gag, env, pol and nef polypeptides and each fragment has a length from about four amino acids to about 400 amino acids.

10. The population of claim 6, wherein said population comprises nucleotide sequences encoding overlapping fragments of the gag, env, pol and nef polypeptides and each fragment has a length from about four amino acids to about 100 amino acids to about 250 amino acids.

11. The population of claim 2, wherein said population comprises nucleotide sequences encoding at least two polypeptides comprising an HIV polypeptide selected from the group consisting of amino acids 2-128 of the HIV gag polypeptide, amino acids 117-248 of the HIV gag polypeptide, amino acids 233-364 of the HIV gag polypeptide, amino acids 362-509 of the HIV gag polypeptide, amino acids 29-146 of the HIV pol polypeptide, amino acids 218-330 of the HIV pol polypeptide, amino acids 290-472 of the HIV pol polypeptide, amino acids 397-530 of the HIV pol polypeptide, amino acids 490-631 of the HIV pol polypeptide, amino acids 597-767 of the HIV pol polypeptide, amino acids 828-981 of the HIV pol polypeptide, amino acids 18-164 of the HIV env polypeptide, amino acids 71-211 of the HIV env polypeptide, amino acids 148-249 of the HIV env polypeptide, amino acids 237-380 of the HIV env polypeptide, amino acids 335-498 of the HIV env polypeptide, amino acids 486-632 of the HIV env polypeptide, amino acids 526-698 of the HIV env polypeptide, amino acids 712-879 of the HIV env polypeptide, amino acids 1-145 of the HIV nef polypeptide, amino acids 126-262 of the HIV nef polypeptide, and amino acids 1-130 of the HIV tat polypeptide.

12. The population of claim 1, wherein said population comprises nucleotide sequences encoding from about 10% to about 25% of the antigenic polypeptides from said organism other than poliovirus.

13. The population of claim 1, wherein said population comprises nucleotide sequences encoding from about 25% to about 50% of the antigenic polypeptides from said organism other than poliovirus.

14. The population of claim 1, wherein said population comprises nucleotide sequences encoding from about 50% to about 90% of the antigenic polypeptides from said organism other than poliovirus.

15. A method of eliciting an immune response in a mammalian host to an antigenic polypeptide, the method comprising administering a first population of live attenuated recombinant poliovirus to a mammalian host, wherein the first population is in a first strain of poliovirus, wherein the first population comprises at least two member viruses, wherein each of said member virus comprises a nucleotide sequence encoding a different antigenic polypeptide, wherein said administering provides for infection of a host cell and expression of the antigenic polypeptides, and wherein expression of the antigenic polypeptides results in induction of an immune response in the host to the antigenic polypeptides.

16. The method of claim 15, wherein said immune response is a mucosal response.

17. The method of claim 15, further comprising administering to a host a second population of live attenuated recombinant poliovirus, wherein the second population of recombinant poliovirus is in a second strain of poliovirus, wherein the second population comprises at least two member viruses, wherein each of said member virus comprises a nucleotide sequence encoding a different antigenic polypeptide, the second population being administered after administration of the first population, wherein an immune response to the antigenic polypeptides is elicited in the host.

18. The method of claim 15, wherein the second population is administered at a time period of from about 1 day to about 1 week after administration of the first population.

19. The method of claim 15, wherein the second population is administered at a time period of from about 1 week to about 4 weeks after administration of the first population.

20. The method of claim 15, wherein the second population is administered at a time period of from about 1 month to about 6 months after administration of the first population.

21. The method of claim 15, wherein said first population is in the Sabin-1 strain of poliovirus and said second population is in the Sabin-2 strain of poliovirus.

22. A population of live attenuated recombinant replication-competent viruses wherein said population comprises at least two member viruses and wherein each of said member viruses comprises a nucleotide sequence encoding a different antigenic polypeptide from a pathogenic organism other than a parent virus from which the recombinant virus was derived, wherein said nucleotide sequence is capable of being expressed in a eukaryotic cell.

23. The population of claim 22, wherein said antigenic polypeptides are included within polyprotein precursors which are proteolytically processed to release said antigenic polypeptides.

24. The population of claim 22, wherein said antigenic polypeptides have a length of from about four to about 400 amino acids.

25. The population of claim 22, wherein the antigenic polypeptides are human immunodeficiency virus (HIV) polypeptides.

26. The population of claim 25, wherein said population expresses at least three different antigenic polypeptides.

27. The population of claim 25, wherein said population expresses at least four different antigenic polypeptides.

28. The population of claim 25, wherein said HIV is HIV type-1.

29. The population of claim 25, wherein said at least two different antigenic polypeptides are selected from the group consisting of gag, env, pol and nef polypeptides from HIV.

30. The population of claim 27, wherein said population comprises nucleotide sequences encoding overlapping fragments of the gag, env, pol and nef polypeptides, and wherein each fragment has a length from about four amino acids to about 400 amino acids.

31. The population of claim 27, wherein said population comprises nucleotide sequences encoding overlapping fragments of the gag, env, pol and nef polypeptides and each fragment has a length from about four amino acids to about 100 amino acids to about 250 amino acids

32. The population of claim 25, wherein said population comprises nucleotide sequences encoding at least two polypeptides comprising an HIV polypeptide selected from the group consisting of amino acids 2-128 of the HIV gag polypeptide, amino acids 117-248 of the HIV gag polypeptide, amino acids 233-364 of the HIV gag polypeptide, amino acids 362-509 of the HIV gag polypeptide, amino acids 29-146 of the HIV pol polypeptide, amino acids 218-330 of the HIV pol polypeptide, amino acids 290-472 of the HIV pol polypeptide, amino acids 397-530 of the HIV pol polypeptide, amino acids 490-631 of the HIV pol polypeptide, amino acids 597-767 of the HIV pol polypeptide, amino acids 828-981 of the HIV pol polypeptide, amino acids 18-164 of the HIV env polypeptide, amino acids 71-211 of the HIV env polypeptide, amino acids 148-249 of the HW env polypeptide, amino acids 237-380 of the HIV env polypeptide, amino acids 335-498 of the HIV env polypeptide, amino acids 486-632 of the HIV env polypeptide, amino acids 526-698 of the HIV env polypeptide, amino acids 712-879 of the HIV env polypeptide, amino acids 1-145 of the HIV nef polypeptide, amino acids 126-262 of the HIV nef polypeptide, and amino acids 1-130 of the HIV tat polypeptide.

33. The population of claim 22, wherein said population comprises nucleotide sequences encoding from about 10% to about 25% of the antigenic polypeptides from said organism other than poliovirus.

34. The population of claim 22, wherein said population comprises nucleotide sequences encoding from about 25% to about 50% of the antigenic polypeptides from said organism other than poliovirus.

35. The population of claim 22, wherein said population comprises nucleotide sequences encoding from about 50% to about 90% of the antigenic polypeptides from said organism other than poliovirus.

36. The population of claim 22, wherein the pathogenicity of said viruses in a host organism is attenuated.

37. The population of claim 22, wherein the virus is a DNA virus.

38. The population of claim 22, wherein the virus is an RNA virus.

39. The population of claim 38, wherein the virus is a picomavirus,

40. The population of claim 39, wherein the virus is a poliovirus.

41. The population of claim 22, wherein the pathogenic organism is a pathogenic virus.

42. The population of claim 41, wherein the pathogenic virus is a human immunodeficiency virus.

43. The population of claim 41, wherein the pathogenic virus is an influenza virus.

44. A composition comprising:
the population according to claim 22; and
a pharmaceutically acceptable carrier.

45. The composition according to claim 44, wherein the composition is an immunogenic composition.

46. A method of inducing an immune response in a subject, comprising administering an effective amount of a replication-competent virus population of claim 22 to the subject.

47. The method of claim 46, wherein said administering comprises administering an effective amount of a first population of replication-competent viruses in a first strain of said replication-competent viruses; and, after a time, administering an effective amount of a second population in a second strain of said replication-competent viruses, the second strain being a different strain than the first strain.

48. A device for administering an immunogenic composition comprising a replication-competent recombinant virus population according to claim 22.

49. The device according to claim 48, wherein said device comprises a container that contains therein an immunogenic composition according to claim 45.

50. The device according to claim 49, wherein said device comprises a syringe and a needle.

51. A method of making a population of live, attenuated, recombinant replication-competent viruses comprising:
a) inserting a plurality of nucleic acids into a genome of a virus or a portion thereof which is capable of replicating in a desired host organism, said plurality of nucleic acids encoding a plurality of antigenic polypeptides from a pathogenic organism, forming a population of recombinant virus constructs; and
b) obtaining a population of live attenuated recombinant replication-competent viruses from said population of recombinant virus constructs.
